(19) United States
(12) Patent Application Publication
(10) Pub. No.: US 2009/0221440 A1
(43) Pub. Date: Sep. 3, 2009

(54) METHODS AND COMPOSITIONS RELATED TO IDENTIFYING PROTEIN-PROTEIN INTERACTIONS

(75) Inventors: Zhiyong Ding, Pearland, TX (US); Gordon Mills, Houston, TX (US)

Correspondence Address: FULBRIGHT & JAWORSKI L.L.P., 600 CONGRESS AVE., SUITE 2400, AUSTIN, TX, 78701, US

(73) Assignee: Board of Regents, the University of Texas System, Austin, TX (US)

(21) Appl. No.: 11/572,088

(22) PCT Filed: Jul. 12, 2005
(86) PCT No.: PCT/US05/24677

§ 371 Date: Dec. 19, 2008

Related U.S. Application Data
(60) Provisional application No. 60/587,178, filed on Jul. 12, 2004.

(51) Int. Cl. C40B 30/06; C40B 40/02; C12N 15/86; C12N 15/85; C12N 5/10

(52) U.S. Cl. 506/10; 506/14; 536/23.1; 435/320.1; 435/325

(57) ABSTRACT

The present invention involves compositions and methods for assessing protein interactions in eukaryotic cells. Certain embodiments involve Retrovirus-Based Molecular Two-Hybrid Screens (ReMTH). ReMTH screens differ from other methods by performing screens in the native cellular hosts without cDNA library construction. Embodiments of the invention use the advantages of tagging endogenous genes with an exon encoding a marker fragment in combination with protein-fragment complementation assays (PCAs), which includes the complementation of at least two marker fragments to form a detectable marker complex. ReMTH vectors insert a nucleotide sequence encoding a first fragment of a marker, such as green fluorescent protein (GFP), into an endogenous gene resulting in expression of random endogenous genes tagged with a first marker fragment forming an endogenous prey protein or prey protein. ReMTH contain cells also express a bait protein that is fused with a second marker fragment. Prey/Bait interaction produces a reconstituted, detectable marker.

This application claims priority to U.S. Provisional Patent application Ser. No. 60/587,178, filed Jul. 12, 2004, which is incorporated herein by reference in its entirety.

The government may own rights in the present invention pursuant to grant numbers PO1 CA64602 and PO1 CA099031 from the National Cancer Institute.

### I. FIELD OF THE INVENTION

The invention generally relates to the field of biochemistry, molecular biology, cell biology, cancer research, and proteomics. More particularly, it concerns compositions and methods for efficient identification of novel protein-protein interactions.

### II. DESCRIPTION OF RELATED ART

Many processes in biology, including transcription, translation, metabolic transduction pathways, and signal transduction pathways, are mediated by non-covalently associated multienzyme complexes. Much of modern biological research is concerned with identifying proteins involved in cellular processes, and determining their functions and interactions with other proteins involved in specific pathways. Further, with rapid advances in genome sequencing projects, there is a need to develop strategies to define protein interactions that make up functional assemblies of proteins (Lander, 1996; Evangelista et al., 1996). Despite the importance of understanding protein assembly in biological processes, there are few convenient methods for studying protein-protein interactions intact mammalian cells (Fromont-Racine et al., 1997; Guarente, 1993). Approaches include the use of chemical crosslinking reagents and resonance energy transfer between dye-coupled proteins.

A powerful and commonly used strategy is the yeast two-hybrid system, which is used to identify novel protein-protein interactions and to examine the amino acid determinants of specific protein interactions (Fromont-Racine et al., 1997; Adams et al., 1991; Chien et al., 1991; Fields and Song, 1989). The approach allows for rapid screening of a large number of clones, including cDNA libraries. Limitations of this technique include the fact that the interaction must occur in a specific context (the nucleus of S. cerevisiae), and generally cannot be used to distinguish induced versus constitutive interactions, and particularly interactions due to post-translational modifications that may not be faithfully recapitulated in yeast.

Recent advances in human genomics research have led to rapid progress in the identification of novel genes. In applications to biological and pharmaceutical research, there is a need to determine the functions of novel gene products; for example, for genes shown to be involved in disease phenotypes. It is in addressing questions of function where genomics-based pharmaceutical research becomes bogged down and there is now the need for advances in the development of simple and automatable functional assays. A first step in defining the function of a protein is to determine its interactions with other proteins in an appropriate context; that is, since proteins make specific interactions with other proteins or other biopolymers as part of functional assemblies, an appropriate way to examine the function of a gene is to determine the physical relationships of the protein derived from the gene with other cellular proteins.

### SUMMARY OF THE INVENTION

Embodiments of the invention include methods for assessing the interaction between a first protein (bait protein) and a second protein (prey protein). Methods of the invention include A method for assessing the interaction between a bait protein and a prey protein, the method comprising the steps of a) obtaining an exon comprising a coding region for a first marker component, wherein the first marker component is combinable with a second marker component to form a detectable marker; b) obtaining a population of cells expressing a selected bait protein that one desires to test for interaction with a prey protein, wherein the bait protein further comprises the second marker component, to form a bait cell population; c) introducing the exon into the genome of the bait cell population, to form a library of cells comprising the exon introduced into the coding region of genes of the genome; and d) assessing the interaction between the bait protein and a prey protein by detecting the formation of the detectable marker in one or more cells. A marker component as used herein refers to a portion or fragment of a marker complex that will be detect by one carrying out the described methods. A marker complex may be at least two complementing fragments that upon association of the marker fragments or association of two proteins such as fluorescent proteins detected by FRET or some similar detection means. Typically, fragmentation of proteins for a protein complementation assay (PCA) is generally based on rational dissection of a polypeptide chain, for exemplary discussions of PCA see U.S. Pat. Nos. 6,270,964, 6,428,951, 6,294,330, 6,897,017; published US Patent Application 2004137528; and PCT publication WO 2004070351, each of which is incorporated herein by reference in its entirety. Typically, the marker complex will produce some detectable signal that is distinct from the signal produced by each marker component alone. A bait protein may be all or part of a protein of interest, of which one may wish to identify prey proteins that interact with the protein of interest, either directly or indirectly.

The methods may further comprise isolating one or more cells in which a detectable marker is formed. In other aspects of the invention, the methods may also comprise characterizing the gene into which the exon coding for a first or second marker fragment has been introduced. In a particular aspect, the exon coding for a first marker fragment, a second marker fragment or both a first and second marker fragment, are encoded by a retroviral vector. In other embodiments, the expression of a polynucleotide encoding either a prey protein, the bait protein, or both a prey protein and the bait protein are under the control of an inducible promoter. Preferably, the inducible promoter is a tetracycline sensitive promoter. The expression of a prey protein may be under the control of a first inducible promoter and expression of the bait protein is under the control of a second inducible promoter. In certain aspects, the first and second inducible promoters are the same or respond to the same stimulus.

A dectable marker may include, but is not limited to an enzyme, a transcription factor, a radioisotope binding protein, a fluorescent protein, or a fluorescent protein complex. In certain aspects, the fluorescent protein is a green fluorescent protein (GFP), cyan fluorescent protein (CFP), blue fluorescent protein (BFP), yellow fluorescent protein (YFP), a red fluorescent protein (RFP) or various combinations thereof. In still further aspects, the detectable marker comprises one or more fluorescent proteins. The detectable marker typically includes a green fluorescent protein (GFP), cyan fluorescent protein (CFP), blue fluorescent protein (BFP), yellow fluorescent protein (YFP), a red fluorescent protein (RFP) or various combinations thereof. Preferably, the detectable marker complex comprises a Cyan Fluorescent Protein and a Yellow Fluorescent Protein. In yet another aspect of the invention, the enzyme is luciferase. Embodiments of the invention also include a transcription factor as marker that controls expression of a second marker. The second marker may be an enzyme, a radioisotope binding protein, or a fluorescent protein. In certain aspects of the invention the detectable marker is detectable by fluorescence, enzymatic activity, FRET, or NMR. In a preferred embodiment, the marker complex is detectable by FRET. In certain aspects, the cells are eukaryotic cells. Preferably, the eukaryotic cells are mammalian cells. More preferably, cells are human cells.

In still further embodiments, the methods further comprise exposing a first and second population of cells to a first and second condition, respectively; and assessing the difference between protein-protein interactions between the first population of cells and the second population of cells. The methods may also include co-cultivating the cells with a population of retrovirus producing cells, wherein the retrovirus encodes the exogenous exon, for example a first, second, or first and second marker fragment.

Embodiments of the invention also include a bait cell population produced by methods that include obtaining a population of cells expressing a selected bait protein that one desires to test for interaction with a prey protein. In another aspect the bait cell further comprises an exon coding a second marker component. Embodiments of the invention also include a library of cells produced by methods including introducing an exond coding for a first, second, or first and second marker fragment into the genome of a bait cell population.

Yet other embodiments include one or more recombinant nucleic acid molecules comprising one or more exon encoding a first marker component, wherein the first marker component is combinable with a second marker component to form a detectable marker, and a polynucleotide sequence encoding a bait protein that one desires to test for interaction with a prey protein, wherein the bait protein further comprises a second marker component.

In certain aspects, a single nucleic acid molecule may comprise i) an exon encoding a first marker component, wherein the first marker component is combinable with a second marker component to form a detectable marker, and ii) a polynucleotide sequence encoding a bait protein that one desires to test for interaction with a prey protein, wherein the bait protein further comprises a second marker component.

Other aspects of the invention include a nucleic acid molecule further defined as a single nucleic acid molecule encoding an exon encoding a first marker component, wherein the first marker component is combinable with a second marker component to form a detectable marker. In still another aspect, the nucleic acid molecule is further defined as a single nucleic acid molecule encoding an exon encoding a polynucleotide sequence encoding a bait protein that one desires to test for interaction with a prey protein, wherein the bait protein further comprises a second marker component. Typically, the exon comprises a splice donor or splice acceptor site. Preferably, the exon comprises a splice donor site. The expression of the polynucleotide sequence encoding the bait protein may be under the control of a constitutive promoter or an inducible promoter. Preferably, the inducible promoter is a tetracycline inducible promoter. In still further aspects, the expression of the polynucleotide sequence comprising the exon may under the control of an inducible promoter, preferably a tetracycline inducible promoter. In further aspects, the bait protein comprises all or part of a transcription factor, a signal transduction molecule, a receptor molecule, or an enzyme. The nucleic acid molecule(s) may further comprising a polynucleotide sequence encoding a selectable marker. Preferably the selectable marker is puromycin resistance gene. In various aspects, the first and second marker component are complementing components of a fluorescent protein, a fluorescent protein complex, luciferase, xanthine-guanine phosphoribosyl transferase (XGPRT), Bleomycin binding protein (BBP), Hygromycin-B-phosphotransferase, L-histidinol NAD+oxydoreductase, Puromycin N-acetyltransferase, dihydrofolate reductase (DHFR), or a transcription factor. The fluorescent protein may be a blue, a cyan, a green, a yellow or a red fluorescent protein. In certain aspects, the association of marker components form a fluorescent protein complex detectable by FRET. The nucleic acid molecule(s) of the invention may be comprised in an vector. Preferably the vector is plasmid or viral vector. More preferably the viral vector that is capable of integration into the genome of a cell, such as a retroviral vector.

In still further embodiments of the invention include a cell comprising a polynucleotide encoding a bait protein that one desires to test for interaction with a prey protein, wherein the bait protein further comprises a second marker component that is combinable with a first marker component to form a detectable marker. The cell may further comprising an exon encoding a first marker component, wherein the first marker component is combinable with a second marker component to form a detectable marker. Typically, the exon encoding the first marker component is randomly inserted into a genome of a cell. Preferably the cell is an eukaryotic cell. More preferably the eukaryotic cell is a mammalian cell and most preferably the cell is a human cell.

In other embodiments, a kit for assessing protein interactions comprising at least one or more of the nucleic acid molecules of the invention suitably aliquoted in a container is contemplated. The kit may further comprise a second isolated nucleic acid comprising a multiple cloning site operably coupled to a nucleic acid sequence encoding a second marker component, wherein a polynucleotide encoding a protein of interest is cloned into the multiple cloning site to produce a nucleic acid encoding a bait protein. The kit may also comprise, but is not limited to, components for RNA isolation, components for DNA amplification, oligonucleotide primers or a combination thereof.

These vectors, and indeed any of the vectors disclosed herein, and variants of the vectors that will be readily recognized by one of ordinary skill in the art, can be used in any of the methods described herein to form any of the compositions producible by these methods.

As used herein, the phrase “tagging an endogenous gene” means inserting a nucleic acid sequence encoding a marker fragment into a genome such that transcription of an endogenous gene results in the production of a fusion protein comprising all or part of the endogenous gene and a marker fragment.

An exon or “exonic sequence” is defined as any transcribed sequence that is present in the mature RNA molecule. The exon in the vector may contain untranslated sequences, for example, a 5′ untranslated region. Alternatively, or in conjunction with the untranslated sequences, the exon may contain coding sequences such as a start codon and open reading frame. The open reading frame can encode naturally occurring amino acid sequences or non-naturally occurring amino acid sequences (e.g., synthetic codons). The open reading frame may also encode a signal secretion sequence, epitope tag, exon, selectable marker, screenable marker, or nucleotides that function to allow the open reading frame to be preserved when spliced to an endogenous gene.

Embodiments discussed in the context of a methods and/or composition of the invention may be employed with respect to any other method or composition described herein. Thus, an embodiment pertaining to one method or composition may be applied to other methods and compositions of the invention as well.

“A” or “an,” as used herein in the specification, may mean one or more than one. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1 illustrates an exemplary GFP′ vector for use in Retrovirus-Based Molecular Two-Hybrid Screens (ReMTH).

FIG. 2 illustrates a scheme for assessing protein-protein interactions using ReMTH.

FIG. 3 illustrates an exemplary one-step vector for use in an alternative embodiment of ReMTH.

FIG. 4 illustrates a scheme for assessing protein-protein interaction using an alternative embodiment of ReMTH.

FIG. 5 illustrates an exemplary CFP vector for use in an alternative embodiment of ReMTH using detection of marker complexes by FRET.

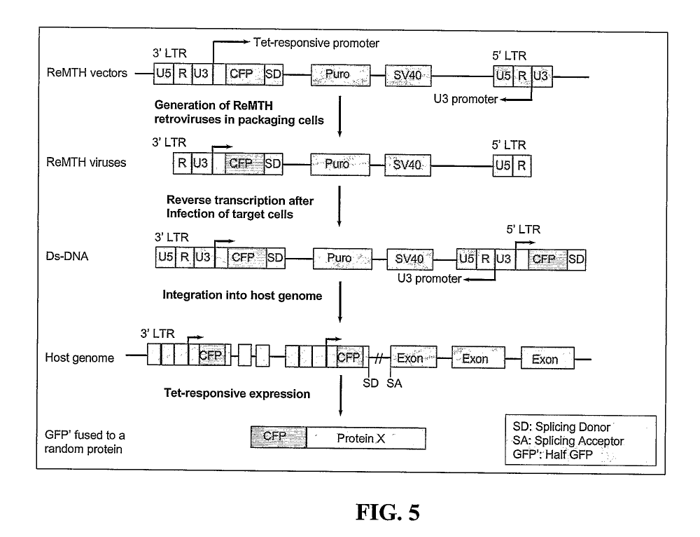

FIG. 6 illustrates a scheme for assessing protein-protein interaction using an alternative embodiment of ReMTH.

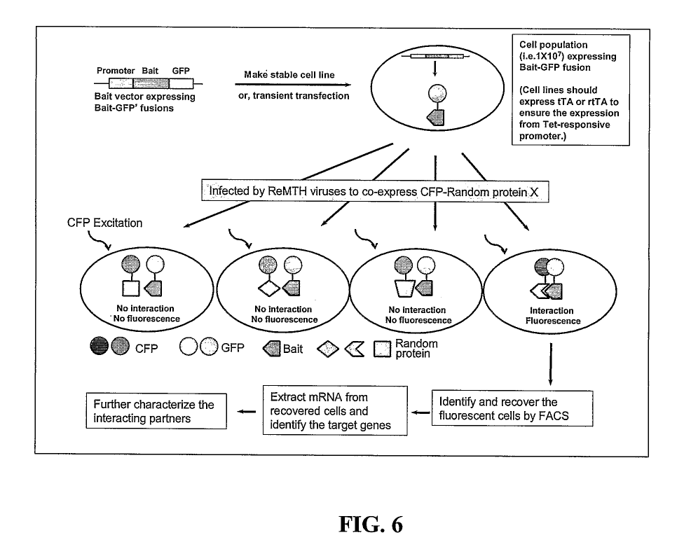

FIG. 7 Schematic diagram of ReMTH screen. (a) Generation of IFP-F[2] (IFPC) fusion to a random endogenous protein in mammalian cells. IFP-F[2] was inserted into ERM retroviral vector, followed by a splice donor, in the U3 region of the 3′ LTR in pBabe-puro. The expression of IFP-F[2] was under the control a tetracycline-responsive promoter. ReMTH retroviruses were generated in packaging cells. After infection of target cells, the retrovirus underwent reverse transcription and integration into the host genome. If the integration occurs upstream or inside of a host gene, the splicing donor in the retroviral element allows the generation of the fusion transcripts of IFP-F[2] to the following exons in the host genome. (b) Procedures of ReMTH screen. Bait is fused to IFP-F[1] (IFPN). Host cell line is transfected to stably express IFP-F[1]-Bait. Host cells should optimally express rTA or rtTA to enable the expression from a tetracycline-responsive promoter. The cell population is infected with ReMTH viruses to co-express the IFP-F[2] endogenous fusion protein. Cells, in which the retrovirus does not generate a fusion protein, or IFP-F[2] is fused to a non-binding partner protein of the bait, are not fluorescent. Only the cells in which IFP-F[2] is fused to an interaction partner of the bait are fluorescent and can be sorted by single cell sorter. The fluorescent cells are expended and the target genes are identified.

FIG. 8 AKT1 interactions with ACTN4 and PDK1. (a) AKT1 interactions with PDK1 and ACTN4 demonstrated by PCA. IFP-F[1]-AKT1 HeLa Tet-on cells were transiently transfected to co-express the IFP-F[2] fusion protein indicated under each image. Co-expression of IFP-F[1]-AKT1 with PDK1-IFP-F[2], or IFP-F[2]-ACTN4wt yielded fluorescence enriched at the leading edge of the cell. Co-expression of IFP-F[1]-AKT1 with IFP-F[2]-ACTN4Δ310-665 yielded cytoplasmic and enhanced nuclear fluorescence. Co-expression of IFP-F[1]-AKT1 with IFP-F[2]-ACTN4Δ310-911 did not result in fluorescence. (b) AKT1 Interactions with ACTN4 demonstrated by co-immunoprecipitation. Upper panel, cells of clone No. 5 expressing IFP-F[1]-AKT1 and IFP-F[2]-ACTN4 were lysed in RIPA buffer. RIPA buffer was used as this was necessary to efficiently release the ACTN4 actin binding protein from cells. Co-IP was performed with anti-AKT1 and western blotting with anti-GFP. Lane 1, IP with normal IgG; lane 2, IP with anti-AKT1; lane 3, total lysate. Lower panel, HeLa Tet-on cells were lysed in RIPA buffer with/without in vivo crosslinking with BASED. Similar results were obtained with DSP crosslinking. Co-IP was performed with anti-AKT1 and western blotting with anti-ACTN4. Lane 1, IP with normal IgG, with crosslinking; lane 2, IP with anti-AKT1, without crosslinking; lane 3, IP with anti-AKT1, with crosslinking; lane 4, total lysate, with crosslinking. (c) Translocation of IFP-F[1]-AKT1 and IFP-F[2]-ACTN4 complex in cells of clone No. 5. The cells were serum starved for 24 hours, followed by serum stimulation for 60 minutes. Serum starved cells showed predominant cytoplasmic fluorescence, while the fluorescence translocated to the leading edge of cells and the periphery of the nucleus after serum stimulation.

FIG. 9 illustrates a clone obtained from a screen with nuclear fluorescence (left panels, target gene not yet identified) and a clone obtained from a screen with cytoplasm fluorescence (right panel, target gene=ACTN4).

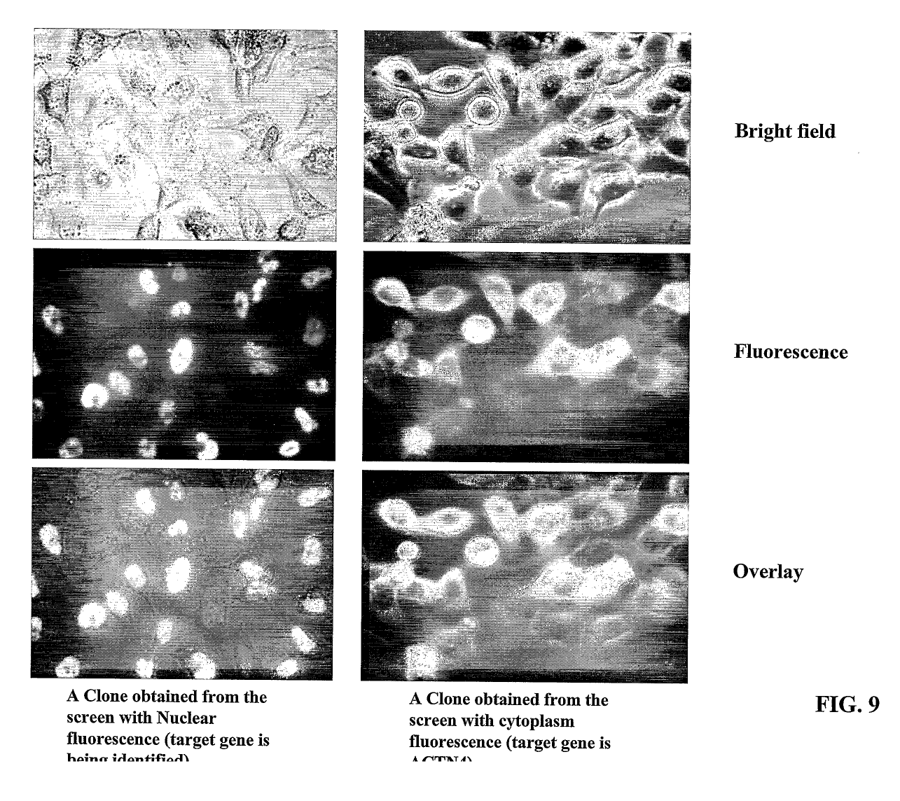

FIG. 10 illustrates a clone obtained from the screen with nuclear fluorescence.

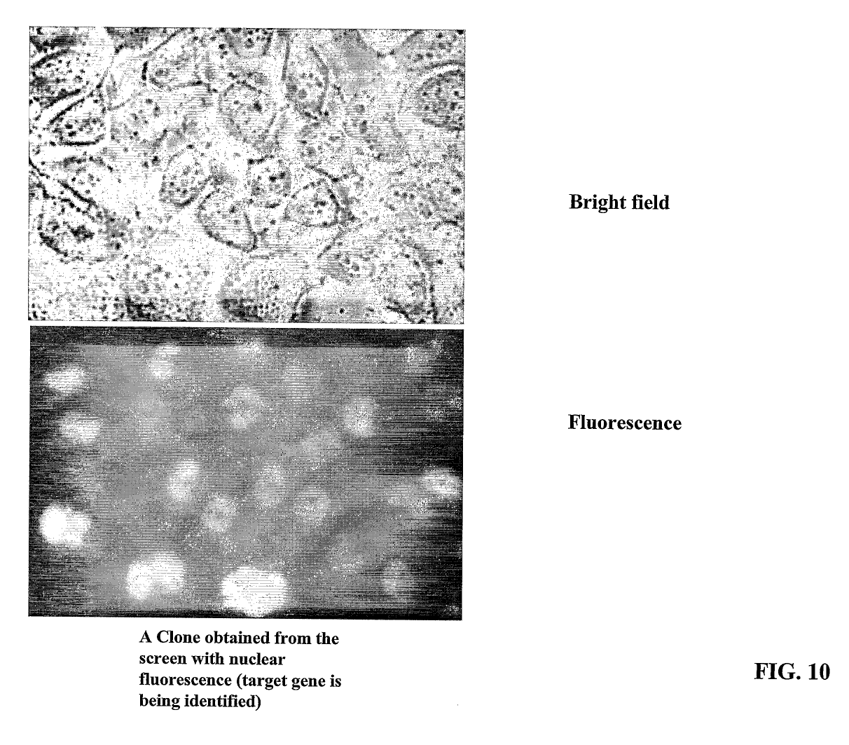

FIG. 11 illustrates a clone obtained from the screen with patterned fluorescence.

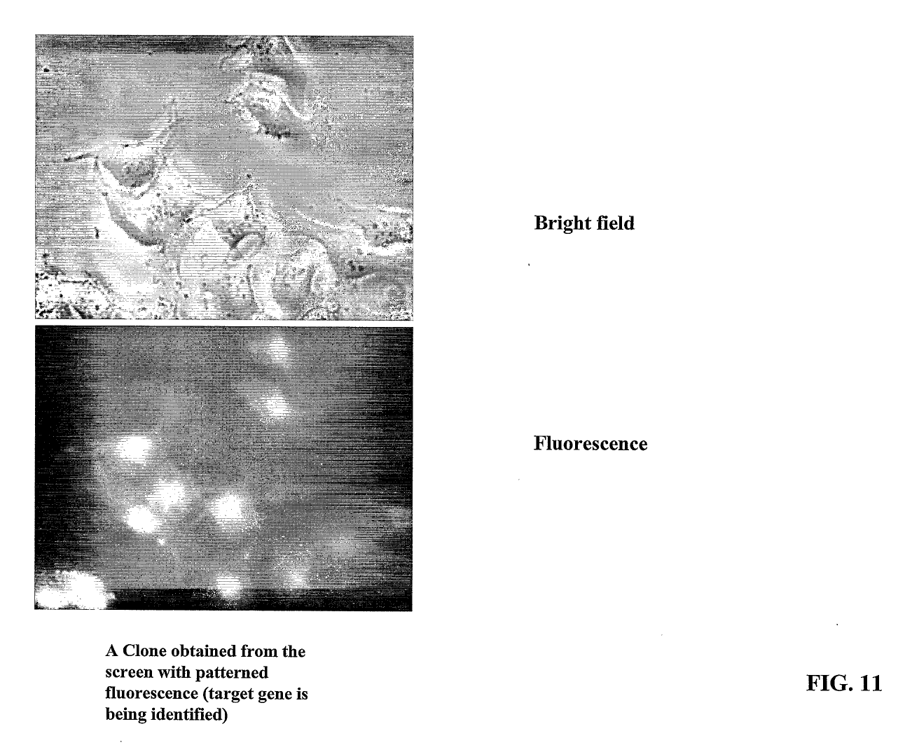

FIG. 12 illustrates a clone obtained from the screen with membrane fluorescence.

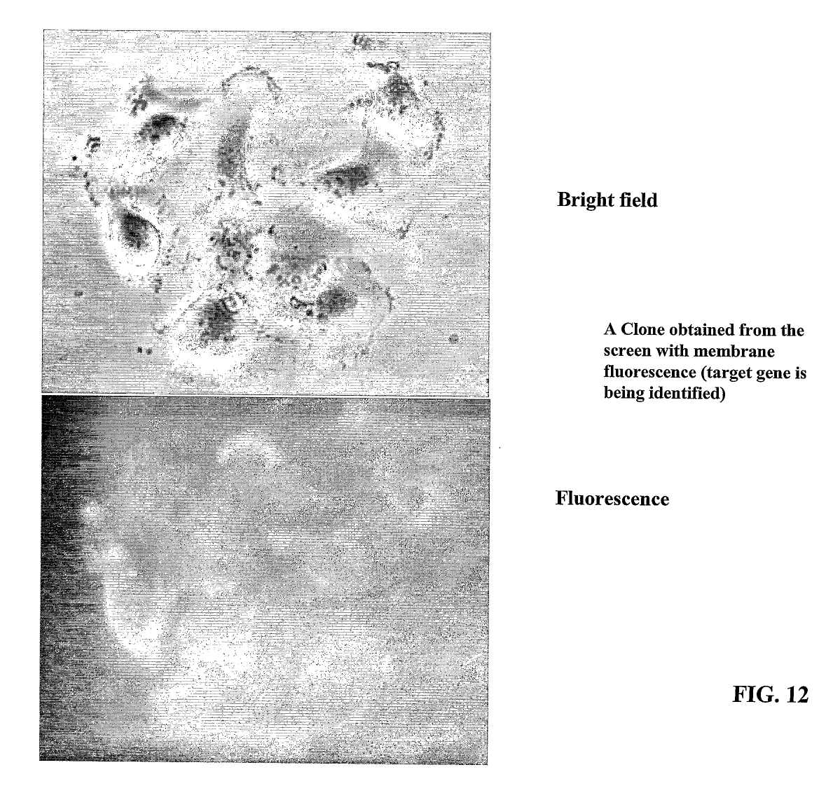

FIG. 13 illustrates a clone obtained from a screen with homogenous fluorescence.

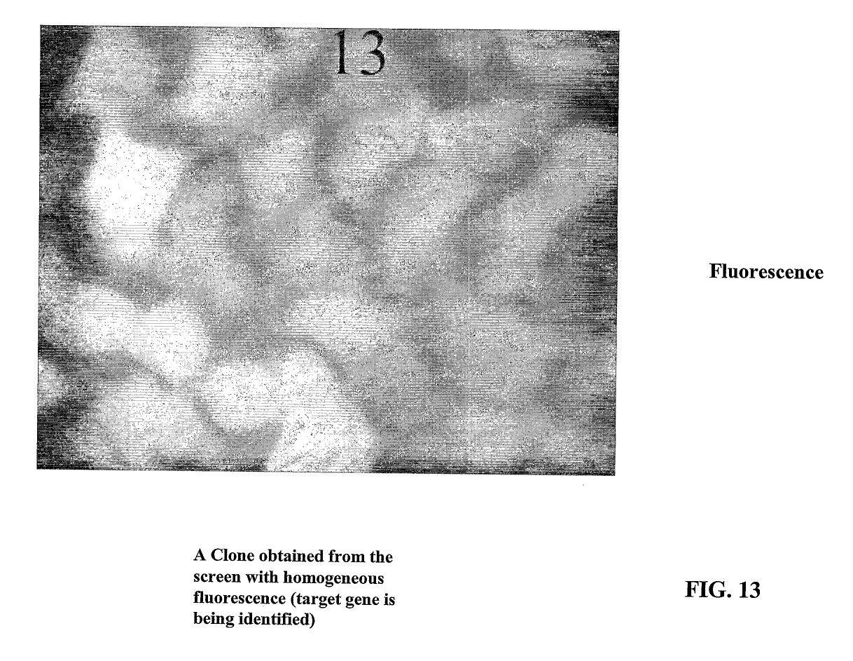

### DESCRIPTION OF ILLUSTRATIVE EMBODIMENTS

In the “post-genome” era, development of methods for the assessment of the functions of a large number of gene products has become more urgent. Most proteins function by interacting with other proteins. An average protein in a human cell may interact with 5 to 15 different proteins individually or in combination. Some pivotal proteins, like Akt, may interact with dozens of different proteins. So, studies of protein interactions are an important part in dissecting cellular functions. Previously described methods for protein interaction screens have contributed to the exploration of new interacting partners for a given protein. However, current methods have many limitations, including but not limited to false protein folding in non-native host or in vitro, failure to undergo faithful post-translational modification in a non-native host and difficulties in the identification of target genes. Many methods have been developed for protein-protein interaction screening purposes, and have contributed greatly to the understanding of protein actions, such as yeast two-hybrid screens, Tap-tag, and co-immunoprecipitation assays. Unfortunately, the limitations of current screens make them inadequate in certain respects to meet the requirements of protein research in this “post-genome” era. The novel methods described herein allow screening of the whole genome for new interactions for any given protein in the context of the endogenous host organism or cell lineage.

Embodiments of the invention include novel screening methods for protein interactions in eukaryotic cells, referred to herein as Retrovirus-Based Molecular Two-Hybrid Screens (ReMTH). ReMTH screens differ from other methods by performing screens in the native cellular hosts without requiring cDNA library construction. Embodiments of the invention use the advantages of enhanced retroviral mutagens (ERM), tagging endogenous genes with an exon encoding a marker fragment, in combination with protein-fragment complementation assays (PCAs), which include the complementation of at least two marker fragments to form a detectable marker complex. ERM was developed to randomly activate endogenous genes throughout the whole genome in mammalian cells by inserting activating exon(s) randomly into endogenous genes. ERM vectors, as well as other gene trapping vectors, have been modified to produce ReMTH vectors by inserting a nucleotide sequence encoding a fragment of a marker, such as green fluorescent protein (GFP), resulting in expression of random endogenous genes tagged with a marker fragment forming the prey protein or endogenous prey protein. When the ReMTH protein products interact with a “bait” protein containing the complementary marker fragment, a functional marker such as GFP is created allowing detection of the protein interaction. ReMTH vector constructs may also contain a promoter element, a regulatable promoter element, a translation start codon, an epitope tag, a sequence encoding a selectable or screenable marker, a sequence encoding a bait protein, or combinations thereof.

Embodiments of the invention include co-expressing a bait protein, which is a fusion protein comprising a protein of interest fused with a marker fragment complementary to that in a ReMTH vector, in a population of cells with the ReMTH vector inserted into the genome of the cells. Co-expression of the bait protein may be accomplished by either introducing a second expression cassette encoding a bait protein into a population of cells carrying an endogenous prey protein or including an expression cassette encoding the bait protein in the ReMTH vector construct used to produce the population of cells expressing the endogenous prey protein. A direct or indirect interaction between a prey protein and the bait protein brings together the first and the second (complementary) fragments of the marker to form a detectable marker, e.g., forming a fluorescent GFP protein. The formation of such a marker is indicative of an interaction taking place directly or indirectly between the prey and the bait. If a prey interacts with the bait, or a molecular complex including the bait, the first and second marker fragment are brought in proximity to each other, restoring detectability of the marker.

One advantage of the invention is a reduced level of false positives relative to other protein interaction assays. False positives are a major problem in the Yeast Two Hybrid system. Many bait proteins for Yeast Two Hybrid can self-activate the reporter gene, making the method unusable. In ReMTH, the bait proteins containing the marker fragment typically behave in the same way as the protein of interest without the marker fragment, thus most if not all proteins can serve as baits. The use of protein complementation in reconstructing a marker molecule provides low false positives and provides a clean background for the ReMTH methods.

The ReMTH methods may be used to identify weak or transient protein-protein interactions. In certain embodiments, ReMTH uses the fluorescence of reconstructed GFP molecules as a readout. GFP molecules have a very compact and tight structure. Once the protein-protein interaction brings the two fragments of GFP together and allows the reconstruction of GFP molecules, approximately in 60 seconds, the two fragments GFP will associate. ReMTH may detect the interactions by detecting the GFP fragments or other reconstituted markers.

Identifying new interacting partners for a given protein using the compositions and methods of the invention will assist in interpreting protein functions, illustrating signaling networks, and determining components of protein complexes. Embodiments of the invention provide a simple, quick, and powerful screening method for the detection of protein interactions in a native cellular host. There are many potential embodiments of the technology including various marker fragments under the control of various promoters with various combinations of baits, expression or expression vectors encoding baits. The methods described herein are both flexible and useful.

The inventive methods typically include introducing a vector containing a nucleic acid sequence encoding a first fragment of a marker (first marker fragment) operably linked to a splice donor sequence into a cell, allowing the vector to integrate into the genome of the cell by non-homologous recombination, expressing a bait protein comprising a second fragment of a marker (second marker fragment) in the cell, and assessing the association of the first and second marker fragments by evaluating a cell for the presence of a reconstituted, detectable marker. The methods do not require any previous knowledge of the sequence of the endogenous gene or even of the existence of the gene. Hence, the invention is directed to the tagging of a random endogenous gene, which as used herein means endogenous gene operably linked to a first marker fragment by non-homologous integration of at least an exon encoding a marker fragment and a splice donor site into the genome of a host cell.

The cell expressing a prey protein may be cultured in vitro under conditions favoring the production of desired amounts of the protein. A cell harboring a prey/bait complex can be detected, identified, and isolated. Cells comprising non-interacting prey/bait proteins will not be detected. Once identified as interacting with the bait protein, the prey protein or the gene encoding the prey protein may be isolated and purified for further use. Genes encoding an endogenous prey protein may be subsequently identified by a variety of nucleic acid cloning and protein isolation techniques including, but not limited to nucleic amplification and direct sequencing of the amplification products, and affinity chromatography, respectfully.

The invention also encompasses a cell or cells comprising a gene encoding a prey protein, a bait expressing vector, or both a gene encoding a prey protein and a bait expressing vector. Expression from the tagged endogenous gene is preferably under the control of a heterologous promoter. In certain aspects, the present invention may also concern regulated expression of an endogenous prey protein. Regulated expression of a prey protein has a number of important applications. First, by expression of genes not normally expressed in a given cell type, it becomes possible to isolate a cDNA copy of genes independent of their normal expression pattern. This facilitates isolation of genes that are normally expressed in rare cells, during short developmental periods, and/or at very low levels. Second, by expressing prey proteins, it is possible to produce protein expression libraries without the need for cloning the full-length cDNA. The methods are capable of identifying new genes that have been or can be missed using conventional and currently available techniques for assessing protein interactions or even for identifying potential protein coding sequences. By using the compositions and methods described herein, unknown and/or uncharacterized protein interactions can be assessed.

The methods can be carried out in any cell of eukaryotic origin, such as fungal, plant or animal. In preferred embodiments, the methods of the invention may be carried out in vertebrate cells, and particularly mammalian cells including, but not limited to, rat, mouse, bovine, porcine, sheep, goat, and human cells, and more preferably in human cells.

A single cell made by the methods described above can express a single prey protein or less preferably more than one prey protein. A cell may express 1, 2, 3, 4, or more prey proteins, preferably one prey protein, as well as 1, 2, 3, 4, or more bait proteins, preferably one bait protein. The invention also encompasses libraries of cells made by the above described methods. A library can encompass the cells from one or more transfections or a subset of clones from one or more transfections. A library can also be formed by combining recombinant cells from two or more transfections, by combining one or more subsets of cells from a single transfection or by combining subsets of cells from separate transfections. Libraries can be formed from the same cell type or different cell types.

### I. Molecular Protein Complementation Screening Methods

Embodiments of the invention include methods for screening for protein-protein interactions in eukaryotic cells, including, but not limited to mammalian cells. In certain aspects, the method may be referred to as retrovirus-based molecular two-hybrid screens (ReMTH). ReMTH utilizes protein complementation of two fragments of a marker protein. A nucleic acid encoding one fragment is associated with a nucleic acid that is non-homologously inserted into the genome of a cell and is associated with an unpaired splice signal (either donor or acceptor). Non-homologous recombination, such as that used by the methods of the present invention, involves the joining (exchange or redistribution) of genetic material that does not share significant sequence homology and does not occur at site-specific recombination sequences. Non-homologous integration of the construct into the genome of a cell results in the operable linkage between the elements of the ReMTH vector and the exons from an endogenous gene. In preferred embodiments, the insertion of the vector is used to tag an endogenous gene and the protein derived from the endogenous gene with a marker fragment. Thus, the present invention uses aspects of protein complementation assay and enhanced retroviral mutagenesis to provide a novel combination of techniques for assessing protein interaction in a cell.

Many different proteins (also referred to herein interchangeably as “gene products” or “expression products”) can be tagged by a construct and in a single set of transfections or infections. Thus, a single cell or different cells in a set of transfectants or infected cells (library) can express more than one protein following transfection with the same or different constructs. Since sequence and structure information is not required for the methods of the present invention, unknown genes may be assessed and identified.

ReMTH is typically performed in various parts of a cell, instead of being limited to the yeast nucleus (Yeast Two Hybrid). This feature enables proteins to fold correctly and to be post-translationally modified in their native hosts. Furthermore, a detectable interaction can take place in any part of the host cell, e.g., cytoplasm, nucleus, or organelles, and be assisted a number of endogenous chaperone proteins and cellular mechanisms. One or the most prominent advantages of this method is the detection of interacting partners of a given protein under native physiological conditions.

No cDNA library construction is needed. ReMTH uses integrating nucleic acids, e.g., retrovirus, to randomly generate marker fragment-tagged proteins in a cell population, and this population of cells with their respective fusion proteins may serve as a library for assessing protein interactions. Furthermore, ReMTH has a limited bias and generates random proteins throughout the whole genome, as compared with Yeast Two Hybrid methods that are bias towards abundant and short cDNAs. Indeed, the propensity of retroviruses to integrate into upstream regulatory regions increases the likelihood of formation of a useful fusion protein including full length proteins.

A Protein-Fragment Complementation Assays (PCAs)

Protein fragment complementation is a general strategy for detecting protein interactions with other biopolymers including other proteins, nucleic acids, carbohydrates or for screening small molecule libraries for compounds of potential therapeutic value. PCA provides an oligomerization-assisted complementation of fragments of monomeric enzymes or other reporter constructs that require no other proteins for the detection of their activity. A PCA has been based on reconstitution of dihydrofolate reductase activity by complementation of defined fragments of the enzyme in E. coli. This assay requires no additional endogenous factors for detecting specific protein-protein interactions (i.e. leucine zipper interactions) and can be extended to screening cDNA, nucleic acid, small molecule or protein design libraries for molecular interactions (for an example see U.S. Pat. No. 6,270,964, which is incorporated herein by reference in its entirety). Alternatively, PCA could be based on reconstitution of two complementary fragments of GFP.

The ReMTH methods have various advantages over PCA-based cDNA library screens. Even though both methods may be performed in native mammalian hosts, there are several significant distinguishing differences conceptually and technically. First, application of PCAs in cDNA library based screen for protein-protein interactions cannot avoid the bias of cDNA libraries towards abundant and short cDNAs. In contrast, ReMTH does not require cDNA libraries, thus providing less biased screens. Second, the PCA cDNA library screen is more technically difficult than ReMTH. PCA contains two steps. In the first step, fluorescence-activated cell sorting (FACS) only collects a pool of positive cells. The target cDNA does not propagate and cannot be identified. In the second step, DNA plasmids are extracted from pooled mammalian cells and transformed into E. coli. Then DNA plasmids were extracted again from individual E. coli clones and verified further. The number of these clones could be very large; therefore, the second step could be laborious. Furthermore, a large portion of DNA plasmids extracted from E. coli clones could be false because they could be co-transfected with true positive plasmids into the same mammalian cell, and then selected by FACS in the first step. In addition, a PCA cDNA screen will miss some targets that may be lethal to a host cell when overexpressed, targets that are expressed at low levels, or targets that are expressed only at particular times or under particular conditions.

In contrast, ReMTH only requires one round of FACS screen. The target genes are propagated in mammalian cells. Furthermore, only one round of RNA extraction and RT-PCR is required to identify target genes and ReMTH has a low false positive rate. In addition, ReMTH may employ regulated promoters, such as Tet-responsive promoters, for regulated expression of endogenous prey and/or bait proteins. The expression level of both bait and prey proteins may be tightly controlled to avoid possible lethal effects.

B Fluorescence Resonance Energy Transfer (FRET)

Fluorescence resonance energy transfer (FRET) is a distance-dependent interaction between the electronic excited states of two molecules, fluorescent proteins, or a combination thereof, in which excitation is transferred from a donor molecule to an acceptor molecule. The efficiency of FRET is dependent on the inverse sixth power of the intermolecular separation, making it useful over distances comparable with the dimensions of biological macromolecules. Thus, FRET is an important technique for investigating a variety of biological phenomena that produce changes in molecular proximity. When FRET is used colocalization of proteins and other molecules can be imaged with spatial resolution beyond the limits of conventional optical microscopy. Conditions for FRET typically include close proximity of donor and acceptor molecules (typically 10-100 Å), and the absorption spectrum of the acceptor must overlap the fluorescence emission spectrum of the donor.

Fluorescent proteins (FPs) like the green-fluorescent-protein (GFP) may be utilized in FRET based methods. They can be genetically fused to bait and prey proteins making them an reporter system for protein-protein interaction in living cells. Several enhanced FP variants with different spectral properties are available. In one example, the cyan-colored CFP as donor and the yellow YFP as acceptor may be used for FRET studies in living cells, since the emission spectrum of CFP partially overlaps the excitation spectrum of YFP. In certain embodiments of the invention, a prey protein may comprise a donor fluorescent protein (e.g., CFP) and bait protein may comprise an acceptor fluorescent protein (e.g., YFP). So, upon interaction between the bait and prey proteins the donor and acceptor fluorescent proteins are brought in proximity to one another so that fluorescence resonance energy transfer may be detected. The exemplary CFP/YFP FRET application requires simultaneous monitoring of spectral emissions at two wavelengths: 480 nm and 535 nm. To allow this simultaneous acquisition of both bands, a High-Efficiency MultiSpec Imager incorporating a 505 nm longpass dichroic filter with emission filters at 480 nm and 535 nm may be used.

C Proteins

Proteins of interest for the production of bait proteins are proteins that may be cloned into an expression vector for the purpose of assessing or screening for direct or indirect protein interactions. Essentially any protein capable of being cloned is a protein of interest and is a candidate for a bait protein of the invention. A protein of interest need not have an identified function or activity. Examples of such proteins include, but are not limited to, cytokines, growth factors, neurotransmitters, enzymes, structural proteins, cell surface receptors, intracellular receptors, hormones, antibodies, and transcription factors to name a few. As one of ordinary skill will appreciate, other cellular proteins and receptors that are known in the art may also be analyzed by ReMTH.

One of the advantages of the method described herein is that virtually any gene encoded in the genome of a cell can be tagged producing a fusion protein comprising all or part of an endogenous gene and a marker fragment, i.e., a prey protein. Constructs of the invention can be transferred (by infection, transduction, transfection, etc.) into cells to produce libraries of prey proteins. Each library contains cells with a unique set of tagged genes.

As noted above, ReMTH provides a powerful approach to discovering and isolating new genes and proteins, and to elucidating the molecular mechanisms used by cells. For some applications of ReMTH, it is desirable to create libraries of cells in which each member of the library contains a vector integrated into various locations in the host cell genome, and in which each member of the library may represent a different tagged endogenous gene.

D Identification and Isolation of Nucleic Acids Encoding Interacting Proteins

Methods and vectors (both DNA and retroviral) are provided for the construction of a library of cells expressing various endogenous prey proteins. The library will preferably contain insertions in essentially all genes or a representative number of genes present in the genome of the cells. The nature of the library and the vectors allow for methods of screening for insertions in specific genes encoding all or part of an endogenous protein that interacts with a protein of interest, and for gathering nucleotide sequence data from each gene identified to provide a database of protein interactions. The invention includes the described library, methods of making the same, and vectors used to construct the library. Accordingly, the invention features a method of producing a cellular library by non-specifically inserting a provirus, e.g., ReMTH vector, into the genomic DNA of a plurality of cells (e.g., human or mouse cells). The vectors of the invention randomly insert into the genome of a cell with a preference for insertion into the 5′ region of genes.

In certain aspects, the ReMTH vectors are a derivative of the ERM vector design. The ERM vectors used were derived from pBabe-puro vector (Morgenstern et al., 1990). In certain embodiments, the ReMTH cassettes contain the tetracycline-regulatable promoter, sequence encoding a marker fragment tag, and a splice donor cloned in the NheI site. This design avoids potential complications arising from the cryptic splice acceptor sequence present in the 3′ end of the 5′LTR. The marker fragment tag encodes a non-functional marker fragment that may be complemented by one or more other marker fragments to reconstitute a detectable marker complex. Examples of related vectors and methods may be found in U.S. Pat. No. 6,207,371 and PCT publication WO 00/53813, both of which are incorporated herein by reference.

In certain aspects, the methods of the invention exploit the structure of the mRNA molecules produced using the integrating vectors of the invention. One such method of the invention comprises, for example, (a) introducing a vector construct comprising an unpaired splice donor site into a host cell preferably a eukaryotic cell), (b) allowing the vector construct to integrate into the genome of the host cell by non-homologous recombination, under conditions such that the vector tags an endogenous gene with an exon encoding a marker fragment producing an endogenous prey protein fusion, (c) introducing into an endogenous prey expressing cell an expression vector encoding a protein of interest fused with a complementing marker fragment, bait fusion protein; (d) selecting a cell in which the prey and bait proteins directly or indirectly interact as indicated by formation of a detectable marker complex; (e) amplifying nucleic acid (DNA or RNA) associated the endogenous gene encoding the prey fusion from a cell. Methods according to this aspect of the invention may comprise one or more additional steps, such as nucleic acid amplification with nested primers. In other embodiments, the integrating vector may also encode the bait protein, thus requiring only a single transduction prior to assessing the formation of detectable marker complexes.

These methods exploit the structure of mRNA molecules produced from the insertion of the marker fragment tag into endogenous genes. The methods of the invention described herein allow virtually any tagged nucleic acid to be isolated, regardless of whether it has been previously isolated and characterized, and regardless of whether it has a known biological activity. This is made possible by the nature of the chimeric transcripts produced from the integrated vectors of the present invention. Each member of the library contains the vector located at various integration site(s), and potentially contains a tagged endogenous gene. Gene tagging occurs when the tagging vector integrates upstream of the 3′-most exon of an endogenous gene and in an orientation capable of allowing transcription from the vector to proceed through the endogenous gene.

In further embodiments, gene tagging occurs when the tagging vector integrates down-stream of the 5′-most exon of an endogenous gene and in an orientation capable of allowing transcription from the vector to proceed through the marker fragment exon. The integration site may be in an intron or exon of the endogenous gene, or may be upstream of the transcription start site of the gene. Following integration, the ReMTH constructs are designed to produce a transcript capable of splicing from an exon encoded by the vector to an exon encoded by an endogenous gene. As a result, a chimeric message is produced that contains the vector exon linked to the exons from an endogenous gene, wherein the endogenous exons are derived from the region located downstream of the vector integration site. For example, the chimeric transcripts can be rapidly isolated to use as probes (to isolate the full length cDNA or genomic copy of the gene or to characterize the gene) or for direct sequencing and/or characterization. In other embodiments, the marker complex or the endogenous prey protein may be isolated and analyzed by peptide sequencing, mass spectrometry or the like.

To isolate the chimeric transcripts activated by vector insertion, RNA is produced from a library member containing an endogenous prey protein. It is also possible to isolate chimeric transcripts from pools of library members in order to increase the through-put of the procedure. cDNA can then be produced from the RNA harvested from the selected cells. Alternatively, total RNA may be used to produce cDNA. In either case, first strand synthesis can be carried out using an oligo dT primer, an oligo dT/poly(A) signal primer, or a random primer. To facilitate cloning of the cDNA product, a poly dT based primer can be used with the structure: 5′-Primer X(dT)₁₋₁₀₀-3′. The oligo dT/poly(A) signal primer can have the structure 5′-(dT)₁₀₋₃₀-Primer X-N₀₋₆-TTTATT-3′. The random primer can have the structure: 5′-(Primer X)NNNNN-3′. In each primer, Primer X is any sequences that can be used to subsequently PCR amplify target nucleic acid molecules. Where the tagged gene product is to be cloned, it is useful to include one or more restriction sites within the primer X sequence to facilitate subsequent cloning. Other primers recognized by those skilled in the art can be used to create first strand cDNA products, including primers that lack a Primer X region. Other methods of nucleic acid analysis such as RACE, Southern blotting, etc. may also be utilitilized (see for example Sambrook, 2001)

In accordance with the invention, the primers may be conjugated with one or more hapten molecules to facilitate subsequent isolation of nucleic acid molecules (e.g., first and/or second strand cDNA products) comprising such primers. After the primer becomes associated with the nucleic acid molecule (via incorporation during cDNA synthesis), selective isolation of the molecule containing the haptenylated primer may be accomplished using a corresponding ligand that specifically interacts with and binds to the hapten via ligand-hapten interactions. In preferred such aspects, the ligand may be bound to, for example, a solid support. Once bound to the solid support, the molecules of interest (haptenylated primer-containing nucleic acid molecules) can be separated from contaminating nucleic acids and other materials by washing the support matrix with a solution, preferably a buffer or water. Cleavage of one or more of the cleavage sites within the primer, or by treatment of the solid support containing the nucleic acid molecule with a high ionic strength elution buffer, then allows for removal of the nucleic acid molecule of interest from the solid support.

Preferred solid supports for use in this aspect of the invention include, but are not limited to, nitrocellulose, diazocellulose, glass, polystyrene, polyvinylchloride, polypropylene, polyethylene, dextran, Sepharose, agar, starch, nylon, latex beads, magnetic beads, paramagnetic beads, superparamagnetic beads or microtitre plates and most preferably a magnetic bead, a paramagnetic bead or a superparamagnetic bead, that comprises one or more ligand molecules specifically recognizing and binding to the hapten molecule on the primer. Hapten molecules for use on the primer molecules of the invention, include without limitation, biotin, an antibody, lipopolysaccharide, and the like.

Following first strand synthesis, second strand cDNA synthesis may be carried out using a primer specific for the vector encoded exon. This creates double stranded cDNA from all transcripts that were derived from the vector encoded promoter. All cellular mRNA (and cDNA) produced from endogenous promoters remains single stranded since the transcript lacks a vector exon at it 5′ end. Once second strand synthesis is carried out, the cDNA may be digested with a restriction enzyme, cloned into a vector, and propagated.

To facilitate cloning, cDNA molecules containing the vector exon may be amplified by PCR using a primer specific for the vector exon and a primer specific for the first strand cDNA primer (e.g., Primer X). PCR amplification results in the production of variable length DNA fragments representing different locations of priming during first strand synthesis and/or amplification of multiple chimeric transcripts from different genes. These amplification products can be sequenced, cloned into plasmids for characterization, or can be labeled and used as a probe.

Other amplification techniques, such as linear amplification using RNA polymerase (Van Gelder, 1990; Eberwine, 1996), can be used. For example, when linear amplification by RNA polymerase is used, a promoter (e.g. T7 promoter) can be placed on the vector exon. As a result, gene activated transcripts will contain the promoter sequence at the 5′ end of the transcript. Alternatively, a promoter can be ligated onto the cDNA molecule following first strand and second strand synthesis. Using either strategy, RNA polymerase is then incubated with cDNA in the presence of ribonucleotide triphosphates to create RNA transcripts from the cDNA. These transcripts are then reverse transcribed to produce cDNA. Since RNA polymerase can create several thousand transcripts from a single cDNA molecule, and since each of these transcripts can be reverse transcribed into cDNA, a large amplification can be achieved. As with PCR, amplification with RNA polymerase can facilitate cloning of activated genes. Other types of amplification strategies are also possible.

In another embodiment, the vector exon containing cDNA molecules are isolated without amplification. This may be useful in instances where biases occur during amplification (for example, when one DNA fragment amplifies more efficiently than another). To produce cDNA enriched for tagged messages, RNA is isolated from the tag library. A primer (e.g., a random hexamer, oligo(dT), or hybrid primers containing a primer linked to poly(dT) or a random nucleotides) is annealed to the RNA and used to direct first strand synthesis. The first strand cDNA molecules are then hybridized to a primer specific for the vector encoded exon. This primer directs second strand synthesis. Following second strand synthesis, the cDNA may be digested with restriction enzymes that cut in the vector exon and in the first strand primer (e.g., in Primer X—see above). The second strand products may then be cloned into a useful vector to allow them to be propagated.

It will be apparent to one of ordinary skill in view of the description contained herein that the cDNA products made according to the methods of the invention may also be cloned into a cloning vector suitable for transfection or transformation of a variety of prokaryotic (bacterial) or eukaryotic (yeast, plant or animal including human and other mammalian) cells. Such cloning vectors, which may be expression vectors, include but are not limited to chromosomal-, episomal- and virus-derived vectors, e.g., vectors derived from bacterial plasmids or bacteriophages, and vectors derived from combinations thereof, such as cosmids and phagemids, BACs, MACs, YACs, and the like. Other vectors suitable for use in accordance with this aspect of the invention, and methods for insertion of DNA fragments therein and transformation of host cells with such cloning vectors, will be familiar to those of ordinary skill in the art.

E Production of cells

A wide variety of host cell lines may be used in conjunction with ReMTH. Any cell, e.g., mammalian cell, that is susceptible to retrovirus infection or permissive for intracellular delivery of an integrating nucleic acid of the invention may be used. In certain embodiments, ReMTH, based on Lentivirus, may be used in non-dividing host cells. Furthermore, the ReMTH system may be modified to performed in a variety of cell lines from insects, birds, amphibians, mollusks, and fish. The methods of the invention may be performed under a variety of physiological conditions. For example, ReMTH performed at different developmental stages or growth conditions may yield different sets of interacting partners, which reflect the physiological relevancy of the interactions.

The constructs of the invention can be integrated into primary, secondary, or immortalized cells. Primary cells are cells that have been isolated from an organism and have not been passaged. Secondary cells are primary cells that have been passaged, but are not immortalized. Immortalized cells are cell lines that can be passaged, apparently indefinitely.

The invention includes eukaryotic host cells comprising one or more of the vector constructs of the invention. Preferred eukaryotic host cells include, but are not limited to, animal cells (including, but not limited to, mammalian (particularly human) cells, insect cells, avian cells, annelid cells, amphibian cells, reptilian cells, and fish cells), plant cells, and fungal cells.

In certain embodiments of the invention, a cell containing an integrated nucleic acid comprising a marker fragment and a splice donor or acceptor, and an expression cassette encoding a bait protein may be assessed for interaction between an endogenous prey protein and the bait protein. The cell expressing an endogenous prey protein can be cultured in vitro under conditions favoring the production of the prey, bait, or prey and bait proteins by the cell.

In certain embodiments, the protein interactions of two or more cell populations may be compared. For example, a parental cell population may be analyzed by the methods of the invention and in addition a genetically modified derivative cell line may be analyzed and compared to the parental, e.g., a parental compared to a knockout cell line. In other embodiments, one cell population may be analyzed and compared to a second cell population being cultivated under different conditions, e.g., one cell population under typical cell culture conditions and a second population under growth stimulating conditions.

F Analysis of Cells

Once a tagged endogenous gene library (or libraries) is created, it can be screened using a number of assays. Depending on the characteristics of the marker proteins or compound used and the nature of the construct used to create the library, any or all of the assays described below can be utilized. Other assay formats can also be used.

FACS assay. The fluorescence-activated cell sorter (FACS) can be used to screen for protein interactions in a number of ways. In a preferred embodiments, the complementation of the marker fragments will provide a fluorescent signal when interacting bait and prey proteins are expressed in the same cell. In other embodiments, a protein complex may be a cell surface complex, then fluorescently-labeled antibodies that bind to a reconstituted marker complex may be incubated with cells expressing bait and prey proteins and assessed for fluorescence. Cells expressing an interacting bait and prey can be sorted according to their fluorescence signal. Fluorescent cells can then be isolated, expanded, and further enriched by FACS, limiting dilution, or other cell purification techniques known in the art.

ELISA. Tagged proteins can be detected using the enzyme-linked immunosorbent assay (ELISA). If the bait/prey complex is secreted, culture supernatants from pools of library cells may be incubated in wells containing bound antibody specific for a reconstituted marker complex. If a cell or group of cells expresses proteins that form bait/prey complexes then the protein will be secreted into the culture media. By screening pools of library clones (the pools can be from 1 to greater than 100,000 library members), pools containing a cell(s) expressing a bait/prey complex can be identified. The cell of interest can then be purified away from the other library members by sib selection, limiting dilution, or other techniques known in the art. In addition to secreted proteins, ELISA can be used to screen for cells expressing intracellular and membrane-bound protein complexes also. In these cases, instead of screening culture supernatants, a small number of cells is removed from the library pool (each cell is represented at least 100-1000 times in each pool), lysed, clarified, and added to the antibody-coated wells.

Magnetic Bead Separation. The principle of this technique is similar to FACS. A membrane bound protein complex is detected by incubating the library with an antibody-conjugated magnetic beads that are specific for the marker complex. If the marker complex is present on the surface of a cell, the magnetic beads will bind to that cell. Using a magnet, the cells expressing the marker complex can be purified away from the other cells in the library. The cells are then released from the beads, expanded, analyzed, and further purified if necessary.

Phenotypic Selection. In this embodiment, cells can be selected based on a phenotype conferred by the marker complex. Examples of phenotypes that can be selected for include growth in a selection medium, motility, immortality or any other selectable phenotype. Isolation of activated cells demonstrating a phenotype, such as described above, is important because the interaction between bait and prey proteins reconstitute the marker complex, which is presumably responsible for the observed cellular phenotype.

The sensitivity of each of the above assays can be effectively increased by upregulating gene expression in the library cells. This can be accomplished by adding an inducer of promoter activity of a bait, a prey, or both a bait and a prey encoding nucleic acid to the cells of the library. These reagents can increase expression of the either the prey, bait or both proteins, thereby allowing a lower sensitivity assay to be used to identify the cell of interest.

In another embodiment, an epitope tag is included in the protein product. The epitope tag can consist of an amino acid sequence that allows affinity purification of the tagged protein (e.g., on immunoaffinity or chelating matrices). Thus, by including an epitope tag on the tagging construct, all of the tagged proteins from a library can be purified. By purifying the tagged proteins away from other cellular and media proteins, screening for novel proteins and enzyme activities can be facilitated. In some instances, it may be desirable to remove the epitope tag following purification of the tagged protein. This can be accomplished by including a protease recognition sequence (e.g., Factor Ia or enterokinase cleavage site) downstream from the epitope tag in the expressed prey protein. Incubation of the purified, activated protein(s) with the appropriate protease will release the epitope tag from the proteins(s).

Once a pool of clones containing cells expressing the a bait/protein complex is identified, steps can be taken to isolate the cell. Isolation of the cell can be accomplished by a variety of methods known in the art. Examples of cell purification methods include limiting dilution, fluorescence activated cell sorting, magnetic bead separation, sib selection, and single colony purification using cloning rings.

Genes that encode membrane associated proteins are particularly interesting from a drug development standpoint. These genes and the proteins they encode can be used, for example, to develop small molecule drugs using combinatorial chemistry libraries and high throughput screening assays. Alternatively, the proteins or soluble forms of the proteins (e.g., truncated proteins lacking the transmembrane region) can be used as therapeutically active agents in humans or animals. Identification of membrane proteins can also be used to identify new ligands (e.g., cytokines, growth factors, and other effector molecules) using ReMTH. Many other uses of membrane proteins are also possible.

Current approaches to identifying genes that encode integral membrane proteins involve isolation and sequencing of genes from cDNA libraries. Integral membrane proteins are then identified by ORF analysis using hydrophobicity plots capable of identifying the transmembrane region of the protein. Unfortunately, using this approach a gene encoding an integral membrane protein can not be identified unless the gene is expressed in the cells used to produce the cDNA library. Furthermore, many genes are only expressed in very rare cells, during short developmental windows, and/or at very low levels. As a result, these genes can not be efficiently identified using the currently available approaches.

The present invention allows an endogenous genes to be tagged without any knowledge of the sequence, structure, function, or expression profile of the genes. The tagged gene may be under the control of a heterologous promoter, in certain aspects the heterologous promoter may be a inducible promoter. A heterologous promoter may be used to express an endogenous at a level and at a time that is amenable to assessment by the inventive methods. Furthermore, using the vectors exemplified herein, the protein produced from the tagged endogenous gene can be modified, for example, to include an epitope tag. These vector can be used to isolate cells that express an integral membrane protein.

### II. Nucleic Acids

In many applications of the invention, it is desirable to produce protein from the tagged endogenous gene, a nucleic acid encoding a bait protein, a nucleic acid encoding a selectable marker or combinations thereof. To accomplish this, a transcriptional regulatory sequence (which may be any transcriptional regulatory sequence, including, but not limited to promoters, enhancers, and repressors) can be placed in the appropriate position upstream or downstream of the nucleic acid(s) to be transcribed and on any of the vectors described herein. To activate expression of full-length protein with the ReMTH vector, however, the vector must integrate into the 5′ UTR, which is typically void of cryptic ATG start codons so as to avoid aberrant translation products from the transcribe gene, of the endogenous gene to avoid cryptic start ATG codons upstream of exon 1. However, if the ReMTH vector is integrated elsewhere it could also produce useful protein.

The transcriptional regulatory sequence on the vector may be operably linked to an exonic sequence, which encodes a first marker fragment, followed by a splice donor site. Additional coding sequence can be located between the translational start codon and the splice donor site. For example, a signal secretion sequence or an epitope tag can be encoded on the vector exon. Transcription regulatory sequences may also be used to drive expression of the nucleic acid sequence encoding a bait protein.

In cases where a start codon is included on the vector exon, it can be advantageous to produce a vector in each reading frame. This is achieved by varying the number of nucleotides between the start codon and the splice donor junction site. Together, the preferred vector configurations are capable of producing protein from endogenous genes, regardless of the exon/intron structure, location of the translation start codon, or reading frame.

In various embodiments, nucleic acids of the invention may comprise a promoter and other nucleic acid elements for the transcription of a nucleic acid encoding an endogenous prey protein or a bait protein. In preferred embodiments, the promoter is a viral promoter. In more preferred embodiments, the viral promoter is the cytomegalovirus immediate early promoter. In alternative embodiments, the promoter is a non-viral promoter or inducible promoter. The transcriptional regulatory sequences used in the vector construct of the invention may also include, but is not limited to, an enhancer. In preferred embodiments, the enhancer is a viral enhancer. In highly preferred embodiments, the viral enhancer is the cytomegalovirus immediate early enhancer. In alternative embodiments, the enhancer is a cellular non-viral enhancer.

The terms upstream and downstream, as used herein, are intended to mean in the 5′ or in the 3′ direction, respectively, relative to the coding strand. The term “upstream region” of a gene is defined as the nucleic acid sequence 5′ of its second exon (relative to the coding strand) up to and including the last exon of the first adjacent gene having the same coding strand. (this may or may not be true based on the concept of multiple ORFs included in a single gene, i.e., p15 and p16). Functionally, the upstream region is any site 5′ of the second exon of an endogenous gene capable of allowing a nonhomologously integrated vector to become operably linked to the endogenous gene.

Splicing of primary transcripts, the process by which introns are removed, is directed by a splice donor site and a splice acceptor site, located at the 5′ and 3′ ends of introns, respectively. The consensus sequence for splice donor sites is (A/C)AG GURAGU (where R represents a purine nucleotide) with nucleotides in positions 1-3 located in the exon and nucleotides GURAGU located in the intron.

An unpaired splice donor site is defined herein as a splice donor site present on the activation construct without a downstream splice acceptor site. When the vector is integrated by nonhomologous recombination into a host cell's genome, the unpaired splice donor site becomes paired with a splice acceptor site from an endogenous gene. The splice donor site from the vector, in conjunction with the splice acceptor site from the endogenous gene, will then direct the excision of all of the sequences between the vector splice donor site and the endogenous splice acceptor site. Excision of these intervening sequences removes sequences that interfere with translation of the endogenous protein.

A Vectors

Vectors of the invention are designed to express a bait protein or non-homologously integrate into the genome of a cell and effect expression of a endogenous prey protein. Non-homologous integration of a construct of the invention into the genome of a cell can tag an endogenous gene with a marker fragment. Expression of the endogenous gene may result in production of full length protein, or in production of a truncated biologically active form of the endogenous protein, depending on the integration site (e.g., upstream region versus an intron or downstream exon). “Biologically active” in the context of this invention includes the ability of the endogenous tagged protein or protein fragment to retain the ability to interact with one or more cellular components. The tagged gene may be a known gene (previously cloned or characterized), the function of which may be known or unknown, or an unknown gene (previously not cloned or characterized).

The term “vector” is used to refer to a carrier nucleic acid molecule into which a second nucleic acid sequence can be inserted for introduction into a cell where it can be replicated or perform its function, such as nucleic acid expression. A nucleic acid sequence can be “exogenous,” which means that it is foreign to the cell into which the vector is being introduced or that the sequence is homologous to a sequence in the cell but in a position within the host cell where the sequence is ordinarily not found. Vectors include retroviruses, plasmids, cosmids, viruses (bacteriophage, animal viruses, and plant viruses), and artificial chromosomes (e.g., YACs). One of skill in the art, in light of the description provided herein, would be well equipped to construct a vector through standard recombinant techniques (see, for example, Maniatis et al., 1990 and Ausubel et al., 1996, both incorporated herein by reference).

The term “expression vector” refers to any type of genetic construct comprising a nucleic acid coding for all or part of a RNA capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

The vectors utilized in the present invention have been engineered to provide unique characteristics applicable to the methods described herein, and to facilitate procedures allowing high throughput. In addition, procedures are described that allow the acquisition of sequence information from each tagged gene or nucleic acid derived therefrom. These latter procedures are also designed for flexibility so that additional molecular information can easily be obtained subsequently. The present invention therefore incorporates gene trapping into a larger and unique tool useful for proteomic and protein interaction assessments.

Gene trapping vectors typically have a splice acceptor preceding a selectable marker and a poly-adenylation signal following the selectable marker, and the selectable marker gene has its own initiator ATG codon. Using this arrangement, the fusion transcripts produced after integration generally only comprise exons 5′ to the insertion site to the known marker sequences. Frequently, gene trapping vectors insert upstream of the first coding exon and therefore contain little if any protein coding information of the gene into which it has inserted. However, vectors have been designed so that 3′ exons are appended to the fusion transcript by replacing the poly-adenylation and transcription termination signals of earlier ROSA vectors with a splice donor (SD) sequence. Consequently transcription and splicing generally results in a fusion between all or most of the endogenous transcript and a marker exon. The exon sequences immediately 3′ to the marker exon may then be sequenced and used to identify the insertion site. The sequence information obtained provides a ready source of probes that may be used to isolate the full-length gene or cDNA from the host cell, or as heterologous probes for the isolation of homologous genes in other species. U.S. Pat. No. 6,207,371, which is incorporated herein in its entirety, describes a gene trapping system based on the published SA (splice acceptor) DNA vectors and the ROSA (reverse orientation, splice acceptor) retroviral vectors (Friedrich and Soriano, 1991 and 1993).

Other vector designs contemplated by the present invention are engineered to include an inducible regulatory elements such as tetracycline, ecdysone, and other steroid-responsive or drug-responsive promoters (No et al., 1996; Furth et al., 1994). These elements are operatively positioned to allow the inducible control of expression of either the selectable marker, the tagged endogenous gene, the nucleic acid encoding a bait protein or combinations thereof, proximal to site of integration. Such inducibility provides a unique tool for the regulation of target gene expression.

All of the vectors are designed to form a fusion transcript between vector encoded sequence and an endogenous gene. To facilitate sequencing, specific sequences may be engineered onto the ends of the marker fragment. Examples of such sequences include, but are not limited to unique sequences for priming PCR, and sequences complementary to standard M13 sequencing primers.

Although specific vectors have been discussed at length above, the invention is by no means to be limited to such vectors. Several other types of vectors may also be used to incorporate engineered exons into cellular transcripts include, but are not limited to, adenoviral vectors, adenoassociated virus vectors, SV40 based vectors, and papilloma virus vectors. Additionally, nucleic acids may also be introduced into the cell by various transfection techniques which are familiar to those skilled in the art such as electroporation, lipofection, calcium phosphate precipitation, infection, retrotransposition, and the like. Examples of such techniques may be found in Sambrook et al. (2001) and Current Protocols in Molecular Biology (1989), all Vols. and periodic updates thereof, herein incorporated by reference. The transfected versions of the retroviral vectors are typically plasmid DNA molecules containing DNA cassettes comprising the described features between the retroviral LTRs.

1. Promoters and Enhancers

The regulatory sequence on a vector can be a constitutive promoter. Alternatively, the promoter may be inducible. Use of inducible promoters will allow low basal levels of activated protein to be produced by the cell during routine culturing and expansion. The cells may then be induced to produce large amounts of the desired proteins, for example, during screening. Examples of inducible promoters include, but are not limited to, the tetracycline inducible promoter and the metallothionein promoter.

The invention also encompasses the use of retrovirus transcriptional regulatory sequences, e.g., long terminal repeats. Where these are used, however, they are not necessarily linked to any retrovirus sequence that materially affects the function of the transcriptional regulatory sequence as a promoter or enhancer of transcription of the endogenous gene to be activated (i.e., the endogenous gene into which the tagging vector integrates).

According to the invention, the transcriptional regulatory sequence (or first or second transcriptional regulatory sequence, in vector constructs having more than one transcriptional regulatory sequence) may be a promoter, a regulated promoter, an enhancer, or a repressor, and is preferably a promoter, including an animal cell promoter, a plant cell promoter, or a fungal cell promoter, most preferably a promoter selected from the group consisting of a CMV immediate early gene promoter, an SV40 T antigen promoter, and a β-actin promoter. Other promoters of animal, plant, or fungal cell origin that may be used in accordance with the invention are known in the art and will be familiar to one of ordinary skill in view of the teachings herein.

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating or inserting an exogenous sequence into the 5′ non-coding sequences located upstream of the coding segment, exon or a combination thereof. Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 2001, incorporated herein by reference). The promoters employed may be constitutive, tissue-specific, inducible, and/or useful under the appropriate conditions to direct expression of the introduced DNA segment, such as is advantageous in the production of recombinant proteins and/or peptides. The promoter may be heterologous or endogenous. Additionally any promoter/enhancer combination (as per, for example, the Eukaryotic Promoter Data Base EPDB, www.epd.isb-sib.ch/) could also be used to drive expression.

The identity of tissue-specific promoters or elements, as well as assays to characterize their activity, is well known to those of skill in the art. Non-limiting examples of such regions include the human LIMK2 gene (Nomoto et al 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), and human platelet endothelial cell adhesion molecule-1 (Almendro et al., 1996).

2. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Samow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

3. Splicing Sites

Most transcribed eukaryotic RNA molecules will undergo RNA splicing to remove introns from the primary transcripts. Vectors containing nucleic acid sequence encoding all or part of a protein sequence (e.g., a marker fragment) may require donor and/or acceptor splicing sites to ensure proper processing of the transcript for protein expression (see, for example, Chandler et al., 1997, herein incorporated by reference) or for tagging an endogenous transcript with a nucleic acid encoding a marker fragment. In a particular embodiment, a nucleic acid sequence encoding a marker fragment is associated with a splice acceptor or splice donor for the purpose of tagging endogenous genes in the genome of a cell.

4. Termination Signals

In certain embodiments, the vectors or constructs of the present invention may comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells.

Terminators contemplated for use in the invention include any known terminator of transcription described herein or known to one of ordinary skill in the art, including but not limited to, the termination sequences of the bovine growth hormone terminator or viral termination sequences, such as the SV40 terminator.

5. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

6. Selectable and Screenable Markers

The selectable marker used in the vector constructs of the invention may be any marker or marker gene that, upon integration of a vector containing the selectable marker into the host cell, either genomically or episomally, permits the selection of a cell containing or expressing the marker gene. Selectable markers include, but are not limited to, a neomycin gene, a hypoxanthine phosphribosyl transferase gene, a puromycin gene, a dihydrooratase gene, a glutamine synthetase gene, a histidine D gene, a carbamyl phosphate synthase gene, a dihydrofolate reductase gene, a multidrug resistance 1 gene, an aspartate transcarbamylase gene, a xanthine-guanine phosphoribosyl transferase gene, an adenosine deaminase gene, and a thymidine kinase gene.

A positive selection marker used in certain aspects of the invention may be any selection marker that, upon expression, produces a protein capable of facilitating the isolation of cells expressing the marker, including but not limited to a neomycin gene, a hypoxanthine phosphribosyl transferase gene, a puromycin gene, a dihydrooratase gene, a glutamine synthetase gene, a histidine D gene, a carbamyl phosphate synthase gene, a dihydrofolate reductase gene, a multidrug resistance 1 gene, an aspartate transcarbamylase gene, a xanthine-guanine phosphoribosyl transferase gene, or an adenosine deaminase gene.

Analogously, a negative selection marker used in these aspects of the invention may be any selection marker that, upon expression, produces a protein capable of facilitating removal of cells expressing the marker, including but not limited to a hypoxanthine phosphribosyl transferase gene, a thymidine kinase gene, or a diphtheria toxin gene.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

To isolate cells that have a tagged endogenous gene, the cells containing the integrated vector can be placed under the appropriate drug selection. Selection for the positive selectable marker and/or against a negative selectable marker can occur simultaneously. In another embodiment, selection can occur sequentially. When selection occurs sequentially, selection for the positive selectable marker can occur first, followed by selection against the negative selectable marker. Alternatively, selection against the negative selectable marker can occur first, followed by selection for the positive selectable marker. A variety of marker fragments may be used in the various aspect of the invention. For example, fluorecent proteins, luciferase, xanthine-guanine phosphoribosyl transferase (XGPRT), Bleomycin binding protein (BBP), hygromycin-B-phosphotransferase, L-histidinol NAD+oxydoreductase, puromycin N-acetyltransferase, dihydrofolate reductase (DHFR). Also contemplated are various cell survival markers, proliferation markers, immortalization proteins, transcription factors that turn on a detectable marker, and circularly permuted GFP that can sense the conformation changes caused by protein-protein interactions to name a few (for examples see U.S. Pat. No. 6,428,951, which is incorporated herein by reference in its entirety).

Marker fragments of many other reporter molecules could be used in place of the exemplary GFP in ReMTH, i.e., dihydrofolate reductase (DHFR) for survival selection, or luciferase for bioluminescent selection. ReMTH provide the first readily and powerful screens performed in the native mammalian cell hosts for protein-protein interactions. There are many potential embodiments of the technology. While the current approach uses PCA with GFP, PCA with any other fluorescent molecule, FRET or use of regulatable promoters are possible and likely. The generalizability of the technology makes it both flexible and useful. (U.S. Pat. No. 6,670,185, incorporated herein by reference in its entirety.)

7. Plasmid Vectors

In certain embodiments, a plasmid vector is contemplated for use to transform a host cell. In preferred embodiments, a plasmid vector is used to express a protein of interest fused with a marker fragment, a bait protein. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Host cells comprising the expression vector are grown in any of a number of suitable media. The expression of the recombinant protein in certain vectors may be induced, as would be understood by those of skill in the art, by contacting a host cell with an agent specific for certain promoters, e.g., by adding tetracycline to the media or by switching incubation to a higher temperature.

8. Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Non-limiting examples of virus vectors that may be used to deliver a nucleic acid of the present invention are described below.

a. Retroviral Vectors

Retroviruses and lentiviruses have promise as delivery vectors in ReMTH due to their ability to integrate their genes into the host genome, transferring a large amount of foreign genetic material, infecting a broad spectrum of species and cell types and of being packaged in special cell-lines (Miller, 1992).

In order to construct a ReMTH retroviral vector, a nucleic acid (e.g., one encoding a marker fragment operably linked to a splice donor or acceptor) is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation), the packaging sequence allows the RNA transcript of the recombinant plasmid to be packaged into viral particles, which are then secreted into the culture media (Nicolas and Rubenstein, 1988; Temin, 1986; Maim et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

b. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

c. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in ReMTH as it has a high frequency of integration and it can infect nondividing cells, thus making it useful for delivery of genes into mammalian cells, for example, in tissue culture (Muzyczka, 1992) or in vivo. Details concerning the generation and use of rAAV vectors are described in U.S. Pat. Nos. 5,139,941 and 4,797,368, each incorporated herein by reference.

d. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

B Vector Delivery and Cell Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

C Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable cell that is capable of replicating a vector and/or expressing a nucleic acid encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting or serially transfecting the host cell with two or more distinct recombinant vectors. Alternatively, a single recombinant vector may be constructed to include multiple distinct coding regions for RNAs, which could then be expressed in host cells transfected with the single vector.

A host cell or cells to be transformed with ReMTH vector(s) may be part of or derived from a tissue or an organism. In certain embodiments, a tissue may comprise, but is not limited to, adipocytes, alveolar, ameloblasts, axon, basal cells, blood (e.g., lymphocytes), blood vessel, bone, bone marrow, brain, breast, cartilage, cervix, colon, cornea, embryonic, endometrium, endothelial, epithelial, esophagus, facia, fibroblast, follicular, ganglion cells, glial cells, goblet cells, kidney, liver, lung, lymph node, muscle, neuron, ovaries, pancreas, peripheral blood, prostate, skin, skin, small intestine, spleen, stem cells, stomach, testes, cancers, anthers, ascite tissue, cobs, ears, flowers, husks, kernels, leaves, meristematic cells, pollen, root tips, roots, silk, stalks, and all cloned, neoplastic or hyperplastic cells thereof.

Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Cell types available for vector replication and/or expression include, but are not limited to, bacteria, such as E. coli (e.g., E. coli strain RR1, E. coli LE392, E. coli B, E. coli X 1776 (ATCC No. 31537) as well as E. coli W3110 (F-, lambda-, prototrophic, ATCC No. 273325), DH5α, JM109, and KC8, bacilli such as Bacillus subtilis; and other enterobacteriaceae such as Salmonella typhimurium, Serratia marcescens, various Pseudomonas specie, as well as a number of commercially available bacterial hosts such as SURE® Competent Cells and SOLOPACK™ Gold Cells (STRATAGENE®, La Jolla). In certain embodiments, bacterial cells such as E. coli LE392 are particularly contemplated as host cells for phage viruses.

Examples of eukaryotic host cells for replication and/or expression of a vector include, but are not limited to, HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

In order to increase integration efficiency and to improve the random distribution of integration sites, cells can be treated with low, intermediate, or high doses of radiation prior to or following transfection. By artificially inducing double strand breaks, the transfected DNA can now integrate into the host cell chromosome as part of the DNA repair process. Normally, creation of double strand breaks to serve as the site of integration is the rate limiting step. Thus, by increasing chromosome breaks using radiation (or other DNA damaging agents), a larger number of integrants can be obtained in a given transfection. Furthermore, the mechanism of DNA breakage by radiation is different than by spontaneous breakage.

It has been shown that DNA repair machinery in the cell can be induced by pre-exposing the cell to low doses of a DNA breaking agent such as radiation or bleomycin. By pretreating cells with these agents approximately 24 hours prior to transfection, the cell will be more efficient at repairing DNA breaks and integrating DNA following transfection. In addition, higher doses of radiation or other DNA breaking agents can be used since the LD50 (the dose that results in lethality in 50% of the exposed cells) is higher following pretreatment. This allows random activation libraries to be created at multiple doses and results in a different distribution of integration sites within the host cell's chromosomes.

### III. Kits

Any of the compositions described herein may be comprised in a kit. In a non-limiting example, a ReMTH vector(s), and/or additional agent, may be comprised in a kit. The kits will thus comprise, in suitable container means, one or more ReMTH vectors, one or more bait expression vectors with cloning sites for a protein of interest and/or various other additional agents.

The kits may comprise a suitably aliquoted vectors and/or additional agent compositions of the present invention, whether labeled or unlabeled, as may be used to prepare a standard curve for a detection assay. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the ReMTH vectors, additional agents, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the kit components are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### ReMTH Experimental Procedures

### Material and Methods

Cell Lines and Plasmids. HeLa Tet-Off and HeLa Tet-On may be purchased from BD Clontech. Packaging cells lines, PT67 and Phoenix (ampho) may be purchased from BD Clontech and Orbigen, respectively. Cells are grown in DMEM supplemented with 10% fetal calf serum. GFP vectors (pEGFP-C1 and pEGFP-N3) are purchased from BD Clontech. The enhanced retroviral mutagen ERM vectors (Liu et al., 2000) were gifts from Dr. Songyang, Department of Biochemistry and Molecular Biology, Baylor College of Medicine, Houston, Tex. There are three ERM vectors, RF1, RF2, and RF3, representing three different reading frames. Six ReMTH vectors are constructed by inserting gene fragments coding for fragments of GFP into above ERM vectors. ReMTH vectors, RF1N, RF2N and RF3N, contain GFP gene amino (N) terminal fragment and generate amino half GFP fused to a random endogenous protein, while RF1C, RF2C and RF3C contain GFP gene carboxy (C) terminal fragment and generate carboxy half GFP fused to a random endogenous protein. Four bait vectors, BV1-BV4, are constructed by substituting full length GFP in pEGFP-C1 or pEGFP-N3 for either N half or C half GFP. This bait vector set may provide ability to clone a given gene fusing to either amino half or carboxy half of GFP, at either its amino terminus or carboxy terminus. Plasmid BV3-Akt1, expressing AKT1-NGFP′, is constructed by inserting Akt1 gene in front of NGFP′ in bait vector BV3. Bait vectors may also be modified to be Tet-responsive.

Preparation of Cells for Bait Expression. Hela Tet-Off is Stably or Transiently transfected with plasmid BV3-Akt1. Expression of the fusion protein, AKT1-NGFP′, is confirmed by western blot using antibodies against AKT1 or GFP. 1-5×10⁷ cells are prepared for screening.

Prepare ReMTH Viruses and Infection. ReMTH vector RF1 C is transiently transfected into Phoenix packaging cells using FuGene6 (Roche). ReMTH vector may direct the production of ReMTH virus RNA within packaging cells. The viruses are collected 48 hours after transfection. HeLa Tet-Off cells expressing bait AKT1-NGFP′ are grown in exponential phase for infection. ReMTH viruses undergo reverse transcription and synthesis of the second DNA strand in HeLa cells. The element containing Tet-responsive promoter, GFP′ and splice donor (SD) is duplicated to 5′ LTR. The following insertion of Ds-DNA format of ReMTH virus into host genome typically results in activating a random endogenous gene and expression of GFP′ fused to a random protein.

FACS Screen and Recovery. The above infected cell population is screened by high-speed FACS 24-36 hours after infection. Alternatively, transduced cell population can be selected by puromycin to enrich positive cells before FACS. Fluorescent cells are collected by single cell sorter and seeded into 96-well plates individually. Cells could be checked by an inverted fluorescence microscope to detect the localization of fluorescence, which reflects the physiological features of the possible interactions. Doxycycline is added to turn off expression from ReMTH promoter in order to avoid the possible lethal effects during cell recovery and expansion. After recovery, clones of cells are examined again for fluorescence by removing Doxycycline to turn on ReMTH promoter expression, this step may provide an additional level of specificity.

Identification of Target Genes. Fusion transcripts containing sequence of GFP′ and an endogenous gene fragment with poly-(A) are generated in recovered cells after ReMTH screen. To identify the target genes, total RNA is extracted from expanded clones using the RNeasy Mini Kit (QIAGEN). Reverse transcription is performed with a random primer RT-1 (5′-GCAAATACGACTCACTATAGGGATCCN(GC)ACG-3′, N=AGCT) (SEQ ID NO:1) (Liu et al., 2000) using Superscript (Invitrogen). The 5′ end of RT-1 primer may contain sequences for the T7 primer. The cDNA is then PCR amplified with specific primers from the GFP′ sequence and the T7 primer using Taq DNA polymerase (Invitrogen). The PCR™ products are then gel purified and directly sequenced. The sequences obtained are used to search the Genebank Non-Redundant and Expressed-sequence-tag (EST) databases using the NCBI Blast program.

Further Characterization of the Partners. After identification of the target genes, full-length cDNA of the genes of interest were obtained by commercial sources or cloning. The interactions may be further confirmed and studied by other genetic or biochemical methods.

### Example 2

### ReMTH Screen in Mammalian Cells Reveals Novel Interaction Partners of PKBA/AKT1

### Methods and Methods

Cell lines and plasmids. HeLa Tet-on cell line was purchased from BD Clontech (Palo Alto, Calif.). 293T/17 cell line was from ATCC (Manassas, Va.). Cells were grown in Dulbecco's modified eagle's medium supplemented with 10% fetal calf serum and antibiotics. Plasmids VYF102 (IFP-F[2] vector), 11117-Y101 (expressing IFP-F[1]-AKT1), and 21622-Y108 (expressing PDK1-IFP-F[2]) were gifts from Odyssey Thera. Inc. (San Ramon, Calif.). The retroviral vector, ERM-R11 was a gift from Dr. Z. Songyang (Baylor College of Medicine, Houston, Tex.). Plasmids pcGP and pVSVG were gifts from Dr. Xiao-Feng Qin (M.D. Anderson Cancer Center). IFP-F[2] was amplified by polymerase chain reaction (PCR) from 21622-Y108 with 5′ primer CTTAATTAAGCCACCATGGGTAAGAACGGCATCAAGGCGAAC (SEQ ID NO:2) and 3′ primer TGGCGCGCCGCTTGTACAGCTCGTCCATGCCGAGAG (SEQ ID NO:3). A ReMTH vector, ReMTH-IFP-F[2]1, was constructed by inserting a PacI/AscI fragment containing the IFP-F[2] coding sequence into the ERM-R1 vector digested with PacI/AscI. ACTN4 cDNA clone was purchased from Origene technologies, Inc. (Rockville, Md.). The ACTN4 coding sequence was PCR amplified with 5′ primer, TGGCGCGCCATGGTGGACTACCACGCGGCGAACC (SEQ ID NO:4), and 3′ primer, ACTCGAGTCACAGGTCGCTCTCGCCATACAAGG (SEQ ID NO:5). The AscI/XhoI PCR fragment was cloned into AscI/XhoI digested VYF 102 to make VYF102-ACTN4wt (IFP-F[2]-ACTN4wt). The plasmid VYF102-ACTN4Δ310-665 was constructed by digestion of VYF108-ACTN4wt with BamHI and re-ligation to delete residue from 310 to 665 (IFP-F[2]-ACTN4Δ310-665). VYF102-ACTN4Δ310-911 was constructed by digestion of VYF102-ACTN4wt with BamHI and XhoI, then re-ligation (IFP-F[2]-ACTN4Δ310-911). All constructs were confirmed by sequencing.

Virus preparation and infection. Retrovirus vectors were prepared as previously described (Soneoka et al., 1995). Briefly, plasmids ReMTH-IFP-F[2]1, pcGP, and pVSVG were transiently transfected into 293T/17 cells using FuGene6 (Roche laboratories, Indianapolis, Ind.). The viruses were collected 48 hours after transfection. Titration and infection were performed as previously described (Soneoka et al., 1995).

ReMTH screen procedure. HeLa Tet-on cells were stably transfected with plasmid 11117-Y111 to express IFP-F[1]-AKT1. IFP-F[1]-AKT1 HeLa Tet-on cells were grown in exponential phase for infection. The infected cells were selected in Dulbecco's modified eagle's medium containing 0.5 μg/ml puromycin (Clontech) for 5 days. In the last 2 days of selection, 2 μg/ml of doxycycline (Clontech) was added to induce the expression from Tet-responsive promoters. Fluorescent cells were sorted individually into 96-well plates. Doxycycline was withdrawn during recovery to reduce the expression of proteins that could have advert effects on cell survival and growth. Ten clones were selected from this round of screening. A set of cells not sorted into 96-well plates were expanded and sorted into 96-well plates. Ten clones were selected from this round of screening.

Identification of target genes. To identify target genes (fish), total RNA was extracted from expanded clones using RNeasy Mini Kits (Qiagen, Valencia, Calif.). Reverse transcription was performed with a random primer RT-1 (5′-GCAAATACGACTCACTATAGGGATCCNNNN(GC)ACG-3′, N=AGCT) (SEQ ID NO:6) (Liu et al., 2000) using Superscript III kit (Invitrogen, Carlsbad, Calif.). The 5′ end of RT-1 primer contains the sequence of T7 primer. The cDNA was then PCR amplified with specific IFP-F[2] primer (IFP-F[2], ACTTCAAGATCCGCCACAACATCGAG) (SEQ ID NO:7) and the T7 primer (T7-2, GCAAATACGACTCACTATAGGGATC) (SEQ ID NO:8) using AccuTaq DNA polymerase (Invitrogen). The PCR products were then gel purified and directly sequenced. The sequences obtained were used to search the Genebank human non-redundant and expressed-sequence-tag databases using Blast programs.

Crosslinking; co-immunoprecipitation, and western blotting. The water-insoluble crosslinker, BASED, was from Pierce Biotechnology, Inc. (Rochford, Ill.). In vivo crosslinking was performed according to manufacturer's suggestions with modifications. Briefly, cells were rinsed by cold PBS twice and treated with 5 mM BASED for 20 minutes. Long wave UV (366 nm) light was used to activate the crosslinking reaction. Cells were lysed in RIPA buffer (Tris-HCl, pH 7.4, 50 mM; NaCl, 150 mM; NP-40, 1%; Sodium deoxycholate, 0.5%; SDS, 0.1%), supplemented with protease inhibitor cocktail (Pierce). Co-IP and western blotting were performed as previously described (Lu et al., 2003). AKT1 antibody for Co-IP was from Santa Cruz Biotechnology, Inc. (Santa Cruz, Calif.). ACTN4 antibody was from Alexis Biochemicals (San Diego, Calif.). GFP antibody was from Abcam (Cambridge, Mass.).

### Results

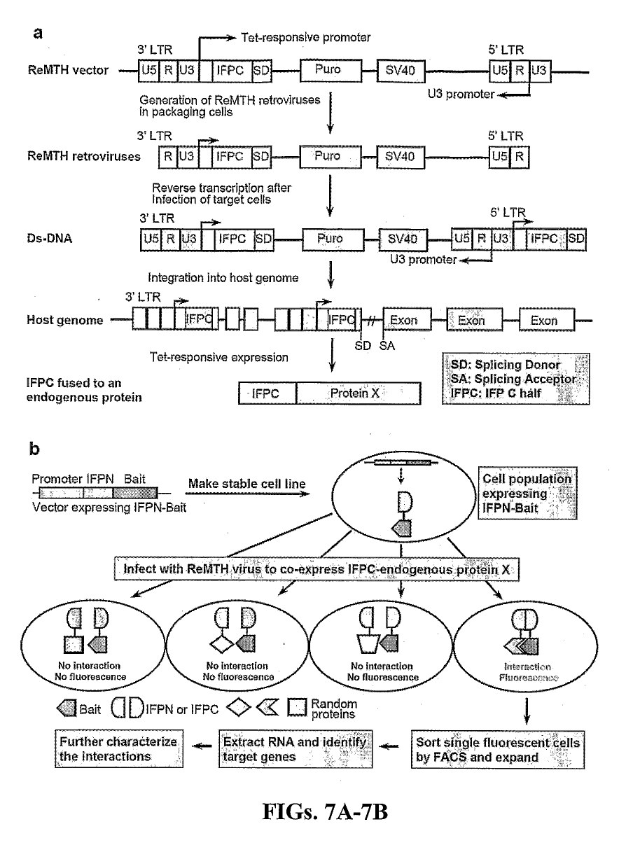

ReMTH (FIG. 7) combines the power of two separate techniques: the ERM exon trap1 and PCA2 providing a facile, sensitive approach to identify novel protein-protein interactions in mammalian cells allowing native protein folding and post-translational modifications while avoiding the bias and difficulty of developing cell specific cDNA libraries. In PCA, GFP, or a related fluorescent molecule, is separated into two non-fluorescent fragments that will not reconstitute spontaneously (Michnick et al., 2000; Ghosh et al., 2000; Hu et al., 2002). When the fragments of GFP is fused to one of a pair of interacting protein partners, the subsequent protein interaction can bring the fragments into proximity creating a stable fluorescent complex.

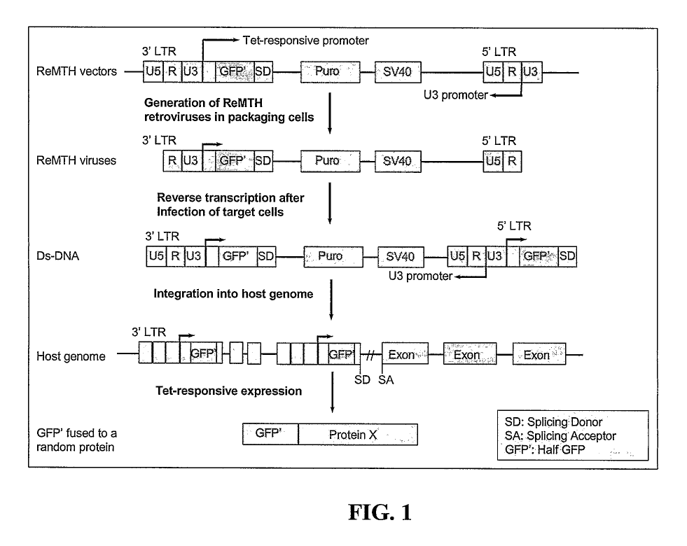

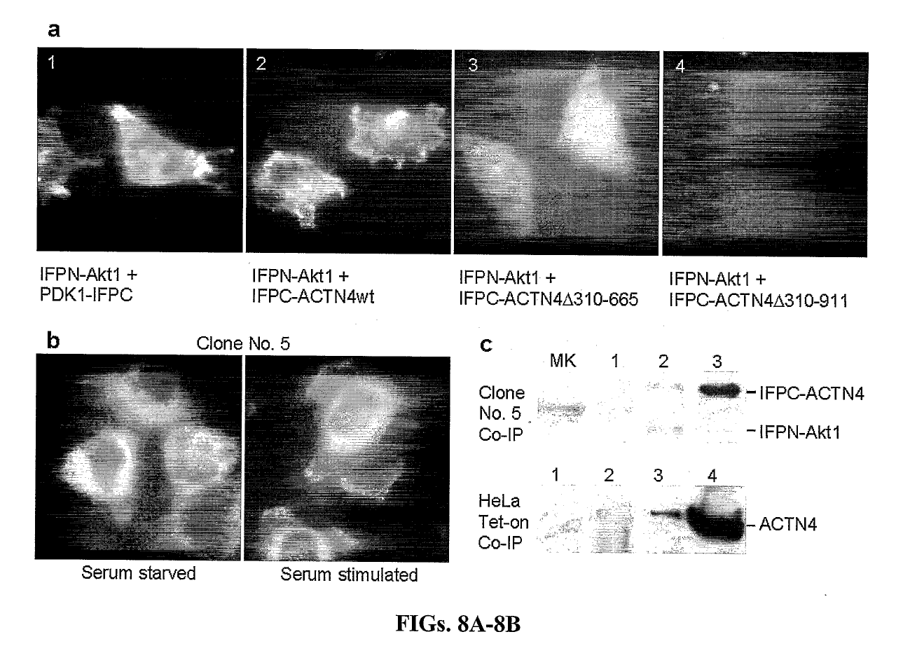

The serine/threonine protein kinase AKT1 (also known as PKBα), which plays a central role in cell metabolism, survival, growth, and tumorigenesis (Braziladn Hemmings, 2001 and 2004), was selected as a bait for a proof of concept of the ReMTH screening approach. The GFP homolog, improved fluorescent protein (IFP), also known as Venus (Nagai et al., 2002), was used as an exemplary reporter. IFP was split into 2 fragments (IFP-F[1] (IFPN) and IFP-F[2] (IFPC)) at residue 158. IFP-F[1]-AKT1 HeLa Tet-on cells (see methods) with or without transient transfection of IFP-F[2] were not fluorescent, confirming that the two fragments of IFP do not fluoresce and further that the two fragments of IFP do not spontaneously associate. Transient transfection of the phosphoinositide-dependent kinase 1 (Alessi et al., 1997) (PDK1)-IFP-F[2] into IFP-F[1]-AKT1 HeLa Tet-on cells resulted in fluorescence, which was enriched at the leading edge of cells (FIG. 8a-1). Thus, linking each fragment of IFP to interacting proteins can reconstitute the fluorescence of IFP with a subsequent appropriate subcellular localization.

IFP-F[1]-AKT1 HeLa Tet-on cells were infected with the ReMTH-IFP-F[2]1 (first open reading frame) retroviral vector. As assessed by western blotting, each of 20 clones, chosen from 2 rounds of single cell sorting, expressed a fusion protein (data not shown) with 13 encoding CAPZB, 3 ACTN4, 2 moesin, and 2 RPL22 (Table 1). Moesin interacts with the tuberous sclerosis complex (TSC) 1 gene product, hamartin (Haddad et al., 2002; Lamb et al., 2000), while AKT phosphorylates the TSC 2 product tuberin (Dan et al., 2002). Because hamartin and tuberin form a tight complex (van Slegtenhorst et al., 1998; Plank et al., 1998), AKT and moesin may associate directly or indirectly in the hamartin:tuberin complex. The interactions of CAPZB and RPL22 with AKT1 do not have precedence. The proof of concept ReMTH screen described herein was performed with IFP-F[1]-AKT1 bait in a single reading frame in HeLa cells under a single culture condition with only a limited number of clones being analyzed. These limitations may contribute to the lack of identification of proteins previously shown to interact with AKT1.

TABLE 1
Potential novel interaction partners
of AKT1 identified by ReMTH.

| | ReMTH | | | | |
|---|---|---|---|---|---|
| | vector | | The amino acid (aa) of | | |
| Clone | reading | Candidates | the target proteins to | | Access |
| ID | frame | identified | which IFP-F[2] fused | | number |
| No. 3 | 1 | CAPZB | 2^{nd} | aa | NP_004921 |
| No. 5 | 1 | ACTN4 | 53^{rd} | aa | NP_004915 |
| No. 22 | 1 | Moesin | 5^{th} | aa | NP_002435 |
| No. 27 | 1 | RPL22 | 5^{tn} | aa | NP_000974 |

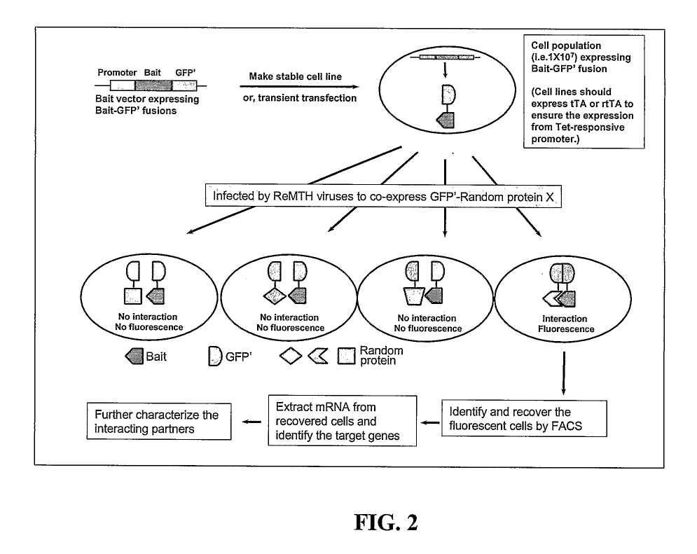

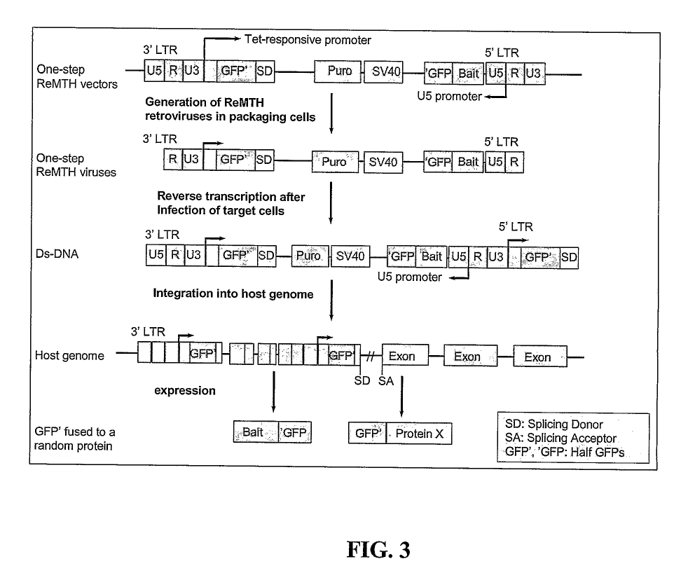

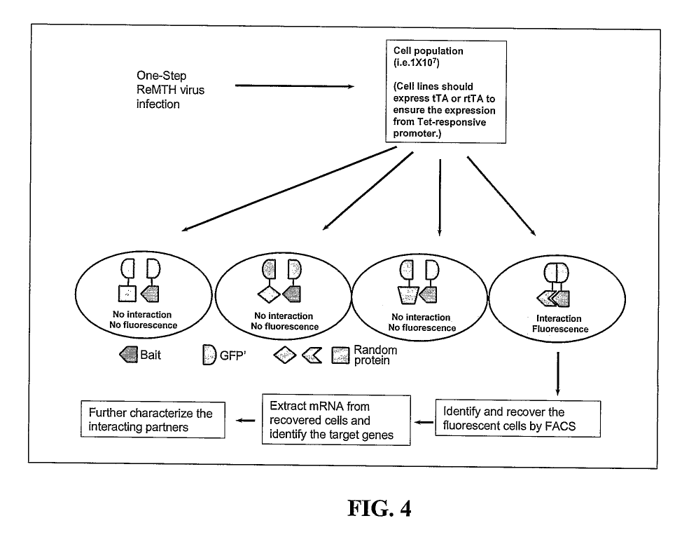

Alpha-actinin actin binding proteins including ACTN4 contain a phosphatidylinositol binding domain, can bind p85 and translocate out of the membrane following inhibition of PI3K (Fukami et al, 1992; Shibasaki et al., 1994; Honda et al., 1998). In addition, ACTN4 contributes to the prognosis of breast cancer (Honda et al., 1998), making it a candidate for further characterization. The AKT1::ACTN4 complex was located throughout the cytoplasm in serum-starved cells (clone No. 5 FIG. 8b). After 60 minutes of serum stimulation, the AKT1::ACTN4 complex translocated to the leading edge of cells and also to the nuclear periphery (FIG. 8b). To identify the residues in ACTN4 required for interaction with AKT1 and localization of the complex to the leading edge of cells, deletion mutants of ACTN4 were created. Expression of the fusion proteins was confirmed by western blotting. Expression of IFP-F[2]-ACTN4wt in IFP-F[1]-AKT1 HeLa Tet-on cells resulted in fluorescence, which was enriched at the leading edge of cells (FIG. 8a-2). In contrast, IFP-F[2]-ACTN4Δ310-665 resulted in homogeneous fluorescence in the cytoplasm with enhanced fluorescence in the nucleus, but completely failed to mediate localization of the fluorescent complex to the leading edge of cells (FIG. 8a-3). Co-expression of IFP-F[1]-AKT1 and IFP-F[2]-ACTN4Δ310-911 did not result in detectable fluorescence (FIG. 8a-4). Thus the C-terminal residues 665-911 of ACTN4, which contains 2 EF motifs, are necessary for the interaction with AKT1, while residues 310-665 of ACTN4 are critical for localization of the AKT1::ACTN4 complex to the leading edge of cells. Strikingly, the interaction of the PH domains of AKT1 with 3-phosphorylated membrane phosphatidylinositols (Stephens et al., 1998) was not sufficient to translocate the AKT1::ACTN4 complex to the cell membrane suggesting that ACTN4 regulates the localization of AKT1.

In cells coexpressing IFP-F[2]-ACTN4 and IFP-F[1]-AKT1 (clone No. 5), IFP-F[2]-ACTN4 was readily immunoprecipitated by anti-AKT1 antibodies and detected by immunoblotting with anti-GFP (FIG. 8c). AKT1 and ACTN4 were present in equal amounts indicating that the majority of the IFP-F[1]-AKT1 was complexed with IFP-F[2]-ACTN4. In parental HeLa Tet-on cells, a stable association between endogenous AKT1 and ACTN4 were unable to be detected, potentially due to the conditions required to efficiently release the actin binding protein ACTN4 (RIPA buffer) from cells disrupting the interaction between AKT1 and ACTN4 or potentially due to the interaction of AKT1 and ACTN4 having a low affinity. However, association between endogenous AKT1 and ACTN4 was detected following in vivo crosslinking (FIG. 8c) demonstrating association of endogenous AKT1 and ACTN4.

A novel ReMTH screen method has been established to detect protein-protein interactions in mammalian cells. The screen is performed in native cell hosts without the need to generate a cell specific cDNA library, which allows native protein folding and posttranslational modifications while avoiding the bias of cDNA libraries. ReMTH has the potential to identify a series of novel context dependent protein::protein interactions in the homologous mammalian environment.

All of the compositions and methods disclosed and claimed herein can be made and executed without undue experimentation in light of the present disclosure. While the compositions and methods of this invention have been described in terms of preferred embodiments, it will be apparent to those of skill in the art that variations maybe applied to the compositions and methods and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by
U.S. Pat. No. 4,683,202
U.S. Pat. No. 4,684,611
U.S. Pat. No. 4,797,368
U.S. Pat. No. 4,952,500
U.S. Pat. No. 5,139,941
U.S. Pat. No. 5,302,523
U.S. Pat. No. 5,322,783
U.S. Pat. No. 5,384,253
U.S. Pat. No. 5,464,765
U.S. Pat. No. 5,538,877
U.S. Pat. No. 5,538,880
U.S. Pat. No. 5,550,318
U.S. Pat. No. 5,563,055
U.S. Pat. No. 5,563,055
U.S. Pat. No. 5,580,859
U.S. Pat. No. 5,589,466
U.S. Pat. No. 5,591,616
U.S. Pat. No. 5,610,042
U.S. Pat. No. 5,656,610
U.S. Pat. No. 5,702,932
U.S. Pat. No. 5,736,524
U.S. Pat. No. 5,780,448
U.S. Pat. No. 5,789,215
U.S. Pat. No. 5,925,565
U.S. Pat. No. 5,928,906
U.S. Pat. No. 5,935,819
U.S. Pat. No. 5,945,100
U.S. Pat. No. 5,981,274
U.S. Pat. No. 5,994,136
U.S. Pat. No. 5,994,624
U.S. Pat. No. 6,013,516
U.S. Pat. No. 6,207,371
U.S. Pat. No. 6,270,964
U.S. Pat. No. 6,428,951
U.S. Pat. No. 6,670,195
Adams et al., Nature, 349:694-697, 1991.
Almendro et al., J. Immunol., 157(12):5411-5421, 1996.
Ausubel et al., In: Current Protocols in Molecular Biology, John, Wiley & Sons, Inc, New York, 1996.
Baichwal and Sugden, In: Gene Transfer, Kucherlapati (Ed.), NY, Plenum Press, 117-148, 1986.
Blomer et al., J. Virol., 71(9):6641-6649, 1997.
Chandler et al., Proc. Natl. Acad. Sci. USA, 94(8):3596-601, 1997.
Chen and Okayama, Mol. Cell Biol., 7(8):2745-2752, 1987.
Chien et al., Proc. Natl. Acad. Sci. USA, 88:9578-82, 1991.
Cotten et al., Proc. Natl. Acad. Sci. USA, 89(13):6094-6098, 1992.
Coupar et al., Gene, 68:1-10, 1988.
Curiel, Nat. Immun., 13(2-3):141-164, 1994.
Eberwine, Methods, 10:283-288, 1996.
Evangelista et al., Trends in Cell Biology, 6:196-199, 1996.
Fechheimer, et al., Proc Natl. Acad. Sci. USA, 84:8463-8467, 1987.
Fields and Song, Nature, 340:245-246, 1989.
Fraley et al., Proc. Natl. Acad. Sci. USA, 76:3348-3352, 1979.
Friedmann, Science, 244:1275-1281, 1989.
Friedrich and Soriano, Genes Dev., 5(9):1513-1523, 1991.
Friedrich and Soriano, Methods Enzymol., 225:681-701, 1993.
Fromont-Racine et al., Nature Genetics, 16:277-282, 1997.
Furth et al., Proc. Natl. Acad. Sci. USA, 91:9302-9306, 1994
Gopal, Mol. Cell Biol., 5:1188-1190, 1985.
Graham and Van Der Eb, Virology, 52:456-467, 1973.
Grunhaus and Horwitz, Seminar in Virology, 3:237-252, 1992.
Guarente, Proc. Natl. Acad. Sci. USA, 90(5):1639-1641, 1993.
Harland and Weintraub, J. Cell Biol., 101(3):1094-1099, 1985.
Horwich et al. J. Virol., 64:642-650, 1990.
Inouye and Inouye, Nucleic Acids Res., 13:3101-3109, 1985.
Kaeppler et al., Plant Cell Reports, 9:415-418, 1990.
Kaneda et al., Science, 243:375-378, 1989.
Kato et al., J. Biol. Chem., 266:3361-3364, 1991.
Kelleher and Vos, Biotechniques, 17(6):1110-7, 1994.
Kraus et al., FEBS Lett., 428(3):165-170, 1998.
Lander, Science, 274:536-539, 1996.
Lareyre et al., J. Biol. Chem., 274(12):8282-8290, 1999.
Lee et al., DNA Cell Biol., 16(11):1267-1275, 1997.
Liu et al., Curr. Biol., 10(19):1233-1236, 2000.
Liu et al., Oncogene, 19(52):5964-5972, 2000.
Macejak and Sarnow, Nature, 353:90-94, 1991.
Maniatis, et al., Molecular Cloning, A Laboratory Manual, Cold Spring Harbor Press, Cold Spring Harbor, N.Y., 1990.
Mann et al., Cell, 33:153-159, 1983.
Miller et al., Am. J. Clin. Oncol, 15(3):216-221, 1992.
Morgenstern et al., Nucleic Acids Res., 18:3587-3596, 1990
Muzyczka, Curr. Topics Microbiol. Immunol., 158:97-129, 1992.
Nabel et al., Science, 244(4910):1342-1344, 1989.
Naldini et al., Science, 272(5259):263-267, 1996.
Nicolas and Rubenstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
No et al., Proc. Natl. Acad. Sci. USA, 93:3345-3351, 1996
Nomoto et al., Gene, 236(2):259-271, 1999.
Omirulleh et al., Plant Mol. Biol., 21(3):415-28, 1993.
Paskind et al., Virology, 67:242-248, 1975.
PCT Appln. WO 00/53813
PCT Appln. WO 94/09699
PCT Appln. WO 95/06128
Pelletier and Sonenberg, Nature, 334(6180):320-325, 1988.
Potrykus et al., Mol. Gen. Genet., 199:183-188, 1985.
Potter et al., Proc. Natl. Acad. Sci. USA, 81:7161-7165, 1984.
Ridgeway, In: Vectors: A survey of molecular cloning vectors and their uses, Rodriguez and Denhardt (Eds.), Stoneham:Butterworth, 467-492, 1988.
Rippe et al., Mol. Cell Biol., 10:689-695, 1990.
Sambrook et al, In: Molecular cloning, Cold Spring Harbor Laboratory Press, Cold Spring Harbor, N.Y., 2001.
Temin, In: Gene Transfer, Kucherlapati (Ed.), NY, Plenum Press, 149-188, 1986.
Tsumaki et al., J. Biol. Chem., 273(36):22861-22864, 1998.
Tur-Kaspa et al., Mol Cell Biol., 6:716-718, 1986.
Van Gelder, Proc. Natl. Acad. Sci. USA, 87:1663-1667, 1990.
Wilson et al., Science, 244:1344-1346, 1989.
Wong et al., Gene, 10:87-94, 1980.
Wu and Wu, Biochemistry, 27:887-892, 1988.
Wu and Wu, J. Biol. Chem., 262:4429-4432, 1987.
Wu et al., Biochem. Biophys. Res. Commun., 233(1):221-226, 1997.
Zhao-Emonet et al., Biochim. Biophys. Acta, 1442(2-3):109-119, 1998.
Zufferey et al., Nat. Biotechnol., 15(9):871-875, 1997.

1. A method for assessing the interaction between a bait protein and a prey protein, the method comprising the steps of:
a) obtaining an exon comprising a coding region for a first marker component, wherein the first marker component is combinable with a second marker component to form a detectable marker;
b) obtaining a population of cells expressing a selected bait protein that one desires to test for interaction with a prey protein, wherein the bait protein further comprises the second marker component, to form a bait cell population;
c) introducing the exon into the genome of the bait cell population, to form a library of cells comprising the exon introduced into the coding region of genes of the genome; and
d) assessing the interaction between the bait protein and a prey protein by detecting the formation of the detectable marker in one or more cells.

2-24. (canceled)

25. A bait cell population produced by step (b) of claim 1.

26. A library of cells produced by step (c) of claim 1.

27. A recombinant nucleic acid comprising:
a) an exon encoding a first marker component, and
b) a polynucleotide sequence encoding a bait protein comprising a binding domain operatively coupled to a second marker component.

28. The nucleic acid of claim 27, wherein the exon comprises a splice donor or splice acceptor site.

29. The nucleic acid of claim 28, wherein the exon comprises a splice donor site.

30. The nucleic acid of claim 27, wherein the expression of the polynucleotide sequence encoding the bait protein is under the control of a constitutive promoter or an inducible promoter.

31. The nucleic acid of claim 30, wherein the inducible promoter is a tetracycline inducible promoter.

32. The nucleic acid of claim 27, wherein the expression of the polynucleotide sequence comprising the exogenous exon is under the control of an inducible promoter.

33. The nucleic acid of claim 32, wherein the inducible promoter is a tetracycline inducible promoter.

34. The nucleic acid of claim 27, wherein the binding domain comprises all or part of a transcription factor, a signal transduction molecule, a receptor molecule, or an enzyme.

35. The nucleic acid of claim 27, further comprising a polynucleotide sequence encoding a selectable marker.

36. (canceled)

37. The nucleic acid of claim 27, wherein the first and second marker component are complementing components of a fluorescent protein, a fluorescent protein complex, luciferase, xanthine-guanine phosphoribosyl transferase (XGPRT), Bleomycin binding protein (BBP), Hygromycin-B-phosphotransferase, L-histidinol NAD+oxydoreductase, Puromycin N-acetyltransferase, dihydrofolate reductase (DHFR), or a transcription factor.

38. The nucleic acid of claim 37, wherein the fluorescent protein is a blue, a cyan, a green, a yellow or a red fluorescent protein.

39. The nucleic acid of claim 27, wherein the association of marker components form a fluorescent protein complex detectable by FRET.

40. The nucleic acid of claim 27, wherein the nucleic acid is comprised in an vector.

41. The nucleic acid of claim 40, wherein the vector is plasmid or viral vector.

42. The nucleic acid of claim 41, wherein the viral vector is a retroviral vector.

43. A cell comprising the nucleic acid of claim 27.

44-51. (canceled)


SEQUENCE LISTING (8 sequences)

SEQ ID NO: 1 - DNA, 32 bp, Artificial Sequence
  Description of Artificial Sequence  Synthetic Primer
gcaaatacgactcactatagggatccngcacg

SEQ ID NO: 2 - DNA, 42 bp, Artificial Sequence
  Description of Artificial Sequence  Synthetic Primer
cttaattaagccaccatgggtaagaacggcatcaaggcgaac

SEQ ID NO: 3 - DNA, 36 bp, Artificial Sequence
  Description of Artificial Sequence  Synthetic Primer
tggcgcgccgcttgtacagctcgtccatgccgagag

SEQ ID NO: 4 - DNA, 34 bp, Artificial Sequence
  Description of Artificial Sequence  Synthetic Primer
tggcgcgccatggtggactaccacgcggcgaacc

SEQ ID NO: 5 - DNA, 33 bp, Artificial Sequence
  Description of Artificial Sequence  Synthetic Primer
actcgagtcacaggtcgctctcgccatacaagg

SEQ ID NO: 6 - DNA, 35 bp, Artificial Sequence
  Description of Artificial Sequence  Synthetic Primer
gcaaatacgactcactatagggatccnnnngcacg

SEQ ID NO: 7 - DNA, 26 bp, Artificial Sequence
  Description of Artificial Sequence  Synthetic Primer
acttcaagatccgccacaacatcgag

SEQ ID NO: 8 - DNA, 25 bp, Artificial Sequence
  Description of Artificial Sequence  Synthetic Primer
gcaaatacgactcactatagggatc